SEQUENCE LISTING (7 sequences)

SEQ ID NO: 1 - DNA, 1329 bp, Human immunodeficiency virus
  CDS (1)..(1329)
atgggtgcgagagcgtcggtattaagcgggggagaattagataaatgggaaaaaattcggttaaggccagggggaaagaaaaaatataagttaaaacatatagtatgggcaagcagggagctagaacgattcgcagtcaatcctggcctgttagaaacatcagaaggctgcagacaaatattgggacagctacagccatcccttcagacaggatcagaagaacttagatcattatataatacagtagcaaccctctattgtgtacatcaaaggatagatgtaaaagacaccaaggaagctttagagaagatagaggaagagcaaaacaaaagtaagaaaaaggcacagcaagcagcagctgcagctggcacaggaaacagcagccaggtcagccaaaattaccctatagtgcagaacctacaggggcaaatggtacatcaggccatatcacctagaactttaaatgcatgggtaaaagtagtagaagaaaaggctttcagcccagaagtaatacccatgttttcagcattatcagaaggagccaccccacaagatttaaacaccatgctaaacacagtggggggacatcaagcagccatgcaaatgttaaaagagactatcaatgaggaagctgcagaatgggatagagtgcatccagtgcatgcagggcctattgcaccaggccaaatgagagaaccaaggggaagtgacatagcaggaactactagtacccttcaggaacaaataggatggatgacaaataatccacctatcccagtaggagaaatctataaaagatggataatcctgggattaaataaaatagtaagaatgtatagccctaccagcattctggacataagacaaggaccaaaggaaccctttagagattatgtagaccggttctataaaactctaagagccgaacaagcttcacaggatgtaaaaaattggatgacagaaaccttgttggtccaaaatgcaaacccagattgtaagactattttaaaagcattgggaccagcagctacactagaagaaatgatgacagcatgtcagggagtggggggacccggccataaagcaagagttttggctgaagccatgagccaagtaacaaatccagctaacataatgatgcagagaggcaattttaggaaccaaagaaagactgttaagtgtttcaattgtggcaaagaagggcacatagccaaaaattgcagggcccctaggaaaaagggctgttggagatgtggaagggaaggacaccaaatgaaagattgcactgagagacaggctaattttttagggaagatctggccttcctac

SEQ ID NO: 2 - protein, 443 aa, Human immunodeficiency virus
MGARASVLSGGELDKWEKIRLRPGGKKKYKLKHIVWASRELERFAVNPGLLETSEGCRQILGQLQPSLQTGSEELRSLYNTVATLYCVHQRIDVKDTKEALEKIEEEQNKSKKKAQQAAAAAGTGNSSQVSQNYPIVQNLQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQDVKNWMTETLLVQNANPDCKTILKALGPAATLEEMMTACQGVGGPGHKARVLAEAMSQVTNPANIMMQRGNFRNQRKTVKCFNCGKEGHIAKNCRAPRKKGCWRCGREGHQMKDCTERQANFLGKIWPSY

SEQ ID NO: 3 - DNA, 3012 bp, Human immunodeficiency virus
  CDS (1)..(3012)
ttttttagggaagatctggccttcctacaagggaaggccagggaattttcttcagagcagaccagagccaacagccccaccagaagagagcttcaggtttggggaggagaaaacaactccctctcagaagcaggagccgatagacaaggaactgtatcctttaacttccctcagatcactctttggcaacgacccctcgtcacaataaggataggggggcaactaaaggaagctctattagatacaggagcagatgatacagtattagaagaaatgaatttgccaggaaaatggaaaccaaaaatgatagggggaattggaggttttatcaaagtaagacagtacgatcagatacctgtagaaatctgtggacataaagctataggtacagtattagtaggacctacacctgtcaacataattggaagaaatctgttgactcagattggttgtactttaaatttccccattagtcctattgaaactgtaccagtaaaattaaagccaggaatggatggcccaaaagttaagcaatggccattgacagaagaaaaaataaaagcattagtagagatatgtacagaaatggaaaaggaagggaaaatttcaaaaattgggcctgaaaatccatacaatactccagtatttgctataaagaaaaaagacagtactaaatggagaaaactagtagatttcagagaacttaataaaagaactcaagacttctgggaagttcagttaggaataccacaccccgcagggttaaaaaagaaaaaatcagtaacagtattggatgtgggtgatgcatacttttcagttcccttagataaagactttagaaagtatactgcatttaccatacctagtataaacaatgagacaccagggattagatatcagtacaatgtgctgccacagggatggaaaggatcaccagcaatattccaaagtagcatgacaaaaatcttagagccttttagaaaacagaatccagacatagttatctatcaatacatggatgatttgtatgtaggatctgacttagaaatagggcagcatagaacaaaaatagaggaactgagacagcatctgttgaggtggggatttaccacaccagacaaaaaacatcagaaagaacctccattcctttggatgggttatgaactccatcctgataaatggacagtacagcctataatgctgccagaaaaagacagctggactgtcaatgacatacagaagttagtgggaaaattgaattgggcaagtcagatttatgcagggattaaagtaaagcagttatgtaaactccttagaggaaccaaagcactaacagaagtaataccactaacagaagaagcagagctagaactggcagaaaacagggagattctaaaagaaccagtacatgaagtatattatgacccatcaaaagacttagtagcagaaatacagaagcaggggcaaggccaatggacatatcaaatttatcaagagccatttaaaaatctgaaaacaggaaagtatgcaaggatgaggggtgcccacactaatgatgtaaaacagttaacagaggcagtgcaaaaagtatccacagaaagcatagtaatatggggaaagattcctaaatttaaactacccatacaaaaggaaacatgggaagcatggtggatggagtattggcaagctacctggattcctgagtgggagtttgtcaatacccctcccttagtgaaattatggtaccagttagagaaagaacccatagtaggagcagaaactttctatgtagatggggcagctaatagggagactaaattaggaaaagcaggatatgttactgacagaggaagacaaaaagttgtctccatagctgacacaacaaatcagaagactgaattacaagcaattcatctagctttgcaggattcgggattagaagtaaacatagtaacagactcacaatatgcattaggaatcattcaagcacaaccagataagagtgaatcagagttagtcagtcaaataatagagcagttaataaaaaaggaaaaggtctacctggcatgggtaccagcacacaaaggaattggaggaaatgaacaagtagataaattagtcagtgctggaatcaggaaagtactatttttgaatggaatagataaggcccaagaagaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatctagaaggaaaaattatcctggtagcagttcatgtagccagtggatatatagaagcagaagttattccagcagagacagggcaggaaacagcatattttctcttaaaattagcaggaagatggccagtaaaaacaatacatacagacaatggcagcaatttcaccagtactacggttaaggccgcctgttggtgggcagggatcaagcaggaatttggcattccctacaatccccaaagtcaaggagtagtagaatctatgaataatgaattaaagaaaattataggacaggtaagagatcaggctgaacaccttaagacagcagtacaaatggcagtattcatccacaattttaaaagaaaaggggggattgggggatacagtgcaggggaaagaatagtagacataatagcaacagacatacaaactaaagaactacaaaagcaaattacaaaaattcaaaattttcgggtttattacagggacaacaaagatcccctttggaaaggaccagcaaagcttctctggaaaggtgaaggggcagtagtaatacaagataatagtgacataaaagtagtgccaagaagaaaagcaaaaatcattagggattatggaaaacagatggcaggtgatgattgtgtggcaagtagacaggatgaggattag

SEQ ID NO: 4 - protein, 1003 aa, Human immunodeficiency virus
FFREDLAFLQGKAREFSSEQTRANSPTRRELQVWGGENNSLSEAGADRQGTVSFNFPQITLWQRPLVTIRIGGQLKEALLDTGADDTVLEEMNLPGKWKPKMIGGIGGFIKVRQYDQIPVEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQYMDDLYVGSDLEIGQHRTKIEELRQHLLRWGFTTPDKKHQKEPPFLWMGYELHPDKWTVQPIMLPEKDSWTVNDIQKLVGKLNWASQIYAGIKVKQLCKLLRGTKALTEVIPLTEEAELELAENREILKEPVHEVYYDPSKDLVAEIQKQGQGQWTYQIYQEPFKNLKTGKYARMRGAHTNDVKQLTEAVQKVSTESIVIWGKIPKFKLPIQKETWEAWWMEYWQATWIPEWEFVNTPPLVKLWYQLEKEPIVGAETFYVDGAANRETKLGKAGYVTDRGRQKVVSIADTTNQKTELQAIHLALQDSGLEVNIVTDSQYALGIIQAQPDKSESELVSQIIEQLIKKEKVYLAWVPAHKGIGGNEQVDKLVSAGIRKVLFLNGIDKAQEEHEKYHSNWRAMASDFNLPPVVAKEIVASCDKCQLKGEAMHGQVDCSPGIWQLDCTHLEGKIILVAVHVASGYIEAEVIPAETGQETAYFLLKLAGRWPVKTIHTDNGSNFTSTTVKAACWWAGIKQEFGIPYNPQSQGVVESMNNELKKIIGQVRDQAEHLKTAVQMAVFIHNFKRKGGIGGYSAGERIVDIIATDIQTKELQKQITKIQNFRVYYRDNKDPLWKGPAKLLWKGEGAVVIQDNSDIKVVPRRKAKIIRDYGKQMAGDDCVASRQDED

SEQ ID NO: 5 - DNA, 501 bp, Homo sapiens
  CDS (1)..(501)
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 6 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 7 - DNA, 11333 bp, recombinant vector
agacagttatcccaatacggtatacaaggagacaatttatcaatttttgtagattcttccaatgaagttgctataaacaggcactctattataggagctagacagttgaatcctatatgcgtagtatctttttatccctttgatccagaacataaagtttttttcgttatatatgttggtagatataaagataagtattgtggaatttcctacgtagctgatagagaagatatgtacaaagttatcaacaggatatacccgtacgttagttgtttttacctcgtatcagatggtataataaattttcatactactcccgtagctaatcacactagaaatattaaaccccttccagttaattattgtaatactttatgtgaaatagtatatgattttgaatatttaaagtttgaacaaggtgttatgtctattccggtgttcatgccttttgtaccaaaacagtttgtatctattatcaatttaccagatgatattctcataacatgtacagcgtccagtaacatagaatacataacacatatagataataaaaagctaaaaagaatacttataataataaaagataaatttctaaagggtactatcatgcaaggtacttttaaaaaagtaaatatcataagacacaagaagtatacatatactataacgtattcttcttttgattgccctaaactagaagatactaagtcatcgctgccaagtacgtgcaataaagccatattagatgggcgtagatatgttacaaaaacttttaatgatacaatataaatggaaatagctagagaaacgctaataacgataggccttactatattagtagtgttattgataataactggattctcgctagtgctaagattaataccgggtgtttatagttcagtatcgaggtcatcatttacagcaggaagaatacttcgttttatggaaatattttctactattatgtttattcctggaataattatattgtacgctgcttatataagaaaaattaaaatgaaaaataattagaatctgaaaatgtcttctggaagcatccatgttattacaggccctatgttttccggtaaaacatcggagctagtaagaagaataaaaagatttatgctatctaactttaaatgtattattattaaacattgtggagataatagatataatgaggatgatataaacaaagtatatactcatgatctattgtttatggaggctacggcatcttctaatctatctgtattagtacctacgctattaaatgatggagttcaggtaataggtatagacgaggctcaattctttctagacatagtagaatttagtgaatccatggctaatttaggtaaaacagttattgtggccgcgcttaacggtgattttaaacgcgaattattcggtaacgtatataagttattatcattagctgaaacagtgtccagtttgacagctatttgcgtgaaatgctattgcgacgcttcgttttctaaacgagttacagaaaataaagaagtaatggatataggtggtaaagataaatacatagccgtgtgtaggaaatgtttttttagtaattaaggggagatctccccatggcccaaagcggggtttgaacagggtttcgctcaggtttgcctgtgtcatggatgcagcctccagaatacttactggaaactattgtaacccgcctgaagttaaaaagaacaacgcccggcagtgccaggcgttgaaaagattagcgaccggagattggcgggacgaatacgacgcccatatcccacggctgttcaatccaggtatcttgcgggatatcaacaacatagtcatcaaccagcggacgaccagccggttttgcgaagatggtgacaaagtgcgcttttggatacatttcacgaatcgcaaccgcagtaccaccggtatccaccaggtcatcaataacgatgaagccttcgccatcgccttctgcgcgtttcagcactttaagctcgcgctggttgtcgtgatcgtagctggaaatacaaacggtatcgacatgacgaatacccagttcacgcgccagtaacgcacccggtaccagaccgccacggcttacggcaataatgcctttccattgttcagaaggcatcagtcggcttgcgagtttacgtgcatggatctgcaacatgtcccaggtgacgatgtatttttcgctcatgtgaagtgtcccagcctgtttatctacggcttaaaaagtgttcgaggggaaaataggttgcgcgagattatagagatccgtcactgttctttatgatctacttccttaccgtgcaataaattagaatatattttctacttttacgagaaattaattattgtatttattatttatgggtgaaaaacttactataaaaagcgggtgggtttggaattagtgatcagtttatgtatatcgcaactaccggcatatggctattcgacatcgagaacattacccacatgataagagattgtatcagtttcgtagtcttgagtattggtattactatatagtatatgtcgggaattcagatccatgcagatcccccctgcccggttattattatttttgacaccagaccaactggtaatggtagcgaccggcgctcagctggaattccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacgggatctagcatggatctagccatttagtatcctaaaattgaattgtaattatcgataataaatggacggatcgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttttgtaatggatcccagctctctcgacgcaggactcggcttgctgaagcgcgcacagcaagaggcgaggggcggcgactggtgagtacgccaatttttgactagcggaggctagaaggagagagagatgggtgcgagagcgtcggtattaagcgggggagaattagataaatgggaaaaaattcggttaaggccagggggaaagaaaaaatataagttaaaacatatagtatgggcaagcagggagctagaacgattcgcagtcaatcctggcctgttagaaacatcagaaggctgcagacaaatattgggacagctacagccatcccttcagacaggatcagaagaacttagatcattatataatacagtagcaaccctctattgtgtacatcaaaggatagatgtaaaagacaccaaggaagctttagagaagatagaggaagagcaaaacaaaagtaagaaaaaggcacagcaagcagcagctgcagctggcacaggaaacagcagccaggtcagccaaaattaccctatagtgcagaacctacaggggcaaatggtacatcaggccatatcacctagaactttaaatgcatgggtaaaagtagtagaagaaaaggctttcagcccagaagtaatacccatgttttcagcattatcagaaggagccaccccacaagatttaaacaccatgctaaacacagtggggggacatcaagcagccatgcaaatgttaaaagagactatcaatgaggaagctgcagaatgggatagagtgcatccagtgcatgcagggcctattgcaccaggccaaatgagagaaccaaggggaagtgacatagcaggaactactagtacccttcaggaacaaataggatggatgacaaataatccacctatcccagtaggagaaatctataaaagatggataatcctgggattaaataaaatagtaagaatgtatagccctaccagcattctggacataagacaaggaccaaaggaaccctttagagattatgtagaccggttctataaaactctaagagccgaacaagcttcacaggatgtaaaaaattggatgacagaaaccttgttggtccaaaatgcaaacccagattgtaagactattttaaaagcattgggaccagcagctacactagaagaaatgatgacagcatgtcagggagtggggggacccggccataaagcaagagttttggctgaagccatgagccaagtaacaaatccagctaacataatgatgcagagaggcaattttaggaaccaaagaaagactgttaagtgtttcaattgtggcaaagaagggcacatagccaaaaattgcagggcccctaggaaaaagggctgttggagatgtggaagggaaggacaccaaatgaaagattgcactgagagacaggctaattttttagggaagatctggccttcctacaagggaaggccagggaattttcttcagagcagaccagagccaacagccccaccagaagagagcttcaggtttggggaggagaaaacaactccctctcagaagcaggagccgatagacaaggaactgtatcctttaacttccctcagatcactctttggcaacgacccctcgtcacaataaggataggggggcaactaaaggaagctctattagatacaggagcagatgatacagtattagaagaaatgaatttgccaggaaaatggaaaccaaaaatgatagggggaattggaggttttatcaaagtaagacagtacgatcagatacctgtagaaatctgtggacataaagctataggtacagtattagtaggacctacacctgtcaacataattggaagaaatctgttgactcagattggttgtactttaaatttccccattagtcctattgaaactgtaccagtaaaattaaagccaggaatggatggcccaaaagttaagcaatggccattgacagaagaaaaaataaaagcattagtagagatatgtacagaaatggaaaaggaagggaaaatttcaaaaattgggcctgaaaatccatacaatactccagtatttgctataaagaaaaaagacagtactaaatggagaaaactagtagatttcagagaacttaataaaagaactcaagacttctgggaagttcagttaggaataccacaccccgcagggttaaaaaagaaaaaatcagtaacagtattggatgtgggtgatgcatacttttcagttcccttagataaagactttagaaagtatactgcatttaccatacctagtataaacaatgagacaccagggattagatatcagtacaatgtgctgccacagggatggaaaggatcaccagcaatattccaaagtagcatgacaaaaatcttagagccttttagaaaacagaatccagacatagttatctatcaatacatggatgatttgtatgtaggatctgacttagaaatagggcagcatagaacaaaaatagaggaactgagacagcatctgttgaggtggggatttaccacaccagacaaaaaacatcagaaagaacctccattcctttggatgggttatgaactccatcctgataaatggacagtacagcctataatgctgccagaaaaagacagctggactgtcaatgacatacagaagttagtgggaaaattgaattgggcaagtcagatttatgcagggattaaagtaaagcagttatgtaaactccttagaggaaccaaagcactaacagaagtaataccactaacagaagaagcagagctagaactggcagaaaacagggagattctaaaagaaccagtacatgaagtatattatgacccatcaaaagacttagtagcagaaatacagaagcaggggcaaggccaatggacatatcaaatttatcaagagccatttaaaaatctgaaaacaggaaagtatgcaaggatgaggggtgcccacactaatgatgtaaaacagttaacagaggcagtgcaaaaagtatccacagaaagcatagtaatatggggaaagattcctaaatttaaactacccatacaaaaggaaacatgggaagcatggtggatggagtattggcaagctacctggattcctgagtgggagtttgtcaatacccctcccttagtgaaattatggtaccagttagagaaagaacccatagtaggagcagaaactttctatgtagatggggcagctaatagggagactaaattaggaaaagcaggatatgttactgacagaggaagacaaaaagttgtctccatagctgacacaacaaatcagaagactgaattacaagcaattcatctagctttgcaggattcgggattagaagtaaacatagtaacagactcacaatatgcattaggaatcattcaagcacaaccagataagagtgaatcagagttagtcagtcaaataatagagcagttaataaaaaaggaaaaggtctacctggcatgggtaccagcacacaaaggaattggaggaaatgaacaagtagataaattagtcagtgctggaatcaggaaagtactatttttgaatggaatagataaggcccaagaagaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatctagaaggaaaaattatcctggtagcagttcatgtagccagtggatatatagaagcagaagttattccagcagagacagggcaggaaacagcatattttctcttaaaattagcaggaagatggccagtaaaaacaatacatacagacaatggcagcaatttcaccagtactacggttaaggccgcctgttggtgggcagggatcaagcaggaatttggcattccctacaatccccaaagtcaaggagtagtagaatctatgaataatgaattaaagaaaattataggacaggtaagagatcaggctgaacaccttaagacagcagtacaaatggcagtattcatccacaattttaaaagaaaaggggggattgggggatacagtgcaggggaaagaatagtagacataatagcaacagacatacaaactaaagaactacaaaagcaaattacaaaaattcaaaattttcgggtttattacagggacaacaaagatcccctttggaaaggaccagcaaagcttctctggaaaggtgaaggggcagtagtaatacaagataatagtgacataaaagtagtgccaagaagaaaagcaaaaatcattagggattatggaaaacagatggcaggtgatgattgtgtggcaagtagacaggatgaggattagaacatggaaaagtttagtaaaacaccatagggtcgactgcagaagcttccatggggagctctttagtgtaataaatttaataaaatattgacaaaatagttaaatgaatatatgaaagtacattatacacggaatggagttcgatattagttcttgcagaatgatatattctgttctcgaacaatatcactttgttactgataatcgttataacaaccataatcaaaaatttagaattatattatactgtttaaaagattctacgataaagaaatatccgtacaggtttgtttctgaaattcactttgtaagatacataattaacaaattcagggggaaaaatctttacaaaattagtatagaagctatagatatatcaaaaggtagacaacaaataatcagaacctaatttttttatcaaaaaattaaaatataaataaaatgaaaaataacttgtatgaagaaaaaatgaacatgagtaagaaacaagtaaaaactcaaagtaaatgtaataataacgcatctagatttacatgcctggatgcggtgca

(19) United States
(12) Patent Application Publication
(10) Pub. No.: US 2008/0044377 A1
(43) Pub. Date: Feb. 21, 2008

(54) Poxvirus Vector Encoding Retrovirus (Eg Hiv) And Cytokine

(75) Inventors: Larry D. Ward, Hawthorn (AU); Helen Thomson, Port Melbourne (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: Virax Development Pty Ltd, Kew (AU)

(21) Appl. No.: 10/575,928

(22) PCT Filed: Oct. 15, 2004
(86) PCT No.: PCT/AU04/01423

§ 371 Date: Mar. 22, 2007

(30) Foreign Application Priority Data
Oct. 15, 2003 (AU) 2003905642
Oct. 16, 2003 (AU) 2003905683

(51) Int. Cl. A61K 39/21; A61K 38/21; A61K 48/00; A61P 31/18; C12N 15/19; C12N 15/48; C12N 15/49

(52) U.S. Cl. 424/85.2; 424/208.1; 424/85.5; 514/44

(57) ABSTRACT

In one embodiment, there is provided a method for treatment or prophylaxis of one or more symptoms of a retrovirus infection such as HIV infection, comprising the administration of poxvirus vector encoding a retrovirus antigen and a cytokine, or a functional homolog, derivative part or analog thereof, in conjunction with anti-retroviral drug therapy wherein said polypeptide and/or cytokine are expressed in a subject and are effective in maintaining a low viral load in a subject for a period of time, for example effectively preventing, reducing or delaying viral rebound during interruption of anti-retroviral drug treatment.

### BACKGROUND OF THE INVENTION

1. Field of the Invention

The present invention relates generally to a recombinant vector and its use in the treatment and/or prophylaxis of retroviral infections and the symptoms associated therewith. More particularly, the present invention provides a recombinant vector for use in conjunction with anti-retroviral drug treatment (ARDT) to modulate viral load in a subject. The present invention specifically relates to a recombinant poxvirus vector expressing a retrovirus antigen and/or a modulatory factor and its use in conjunction with anti-HIV retroviral drug therapy in the treatment or prophylaxis of HIV infection, AIDS and AIDS-related disorders in a human subject. The vectors and methods of the present invention are particularly useful in preventing, reducing or delaying viral rebound when retroviral therapy is interrupted.

2. Description of the Prior Art

Bibliographic details of the references in this specification are collected at the end of the description.

Reference to any prior art in the specification is not, and should not be taken as, an acknowledgment or any form of suggestion that this prior art forms part of the common general knowledge in any country.

Retroviruses are obligate intracellular parasites of vertebrate cells. Viral propagation of the enveloped RNA virus is via a double stranded DNA provirus intermediate which integrates into the genomic DNA of a susceptible host cell and makes use of many host cell factors. This efficient system of infection and propagation makes eradication of the virus very difficult. It is estimated that HIV replication in an infected individual can involve the production and clearance of 10 billion virions per day, each virion having a half-life of about six hours in the general circulation (Australian Society for HIV Medicine (ASHM)-2001 Australian Antiretroviral Guidelines).

All retrovirus genomes comprise three major coding domains: gag, which is responsible for matrix, capsid and nucleoprotein structures; pol which encodes RNA-dependent DNA polymerase, reverse transcriptase, and also integrase enzymes; and env which generates viral envelope proteins. In addition, all retroviruses also comprise the pro coding domain responsible for producing virion protease. A subset of retroviruses, termed “complex” retroviruses, also comprise a range of regulatory factors which influence their own and host expression pathways.

The retrovirus family includes Lentiviruses such as Human immunodeficiency virus (HIV-1 and HIV-2), Simian immunodeficiency virus (SIV), Human T-cell leukaemia-bovine leukaemia viral group such as Human T-cell leukaemia virus (HTLV), Feline leukaemia virus (FIV) and Spumaviruses as described in Vogt P. K. (Chapter 1: Retroviruses: Coffin John M. et al (eds), Cold Spring Harbour Laboratory Press, USA, 1997).

HIV is a particularly important complex retrovirus of humans as the causative agent of Acquired Immune Deficiency Syndrome (AIDS) which remains a devastating and complex problem despite recent advances in anti-retroviral drug treatments.

HIV infects CD4+ immune cells and established HIV infections are characteristically associated with progressive immune system damage, opportunistic infections and wasting syndromes. Commencement of anti-retroviral therapy is generally recommended at any stage of HIV infection when immune deficiency is present as determined by, for example, low levels of CD4+ cells. Reductions in plasma viral load in response to anti-retroviral treatment are associated with statistically significant improvements in survival and clinical outcome (Melors J. W. et al, Science 272:1167-1170, 1996). Complete eradication of HIV in a subject is presently considered to be an unrealistic goal, and as viral levels may increase or rebound if treatment is discontinued, infected individuals are prima facie committed to a life time of antiretroviral drug treatment.

There are a large range of anti-retroviral drug treatment regimens generally involving the administration of combinations of anti-retroviral compounds (see for example ASHM-2001 draft Australian antiretroviral guidelines, supra). In particular, limited clinical data have indicated that triple therapy in the treatment of acute and advanced HIV infection employing a nucleoside analogue combination and a non-nucleoside reverse transcriptase inhibitor or protease inhibitor has a positive effect on surrogate markers of disease progression and at least a short term clinical benefit.

The optimal regimens and timing for anti-retroviral treatment are unclear. The emergence of drug resistant strains is a major problem contributing to drug treatment failure. Compliance is also a major problem because anti-retroviral drug treatment regimens are characteristically complex and require strict adherence in order to have any chance of success. Current regimens often involve multiple dosings of up to four different active agents. Each active agent typically has its own administration requirements, for example administration before or after food. Similarly each agent will need to be administered in specified quantities at specified periods, such that the patient will frequently be taking, for example, one medication 4 times a day, another 3 times a day and another twice a day, with one needing to be taken before food and one needing to be taken after food. In addition the common side effects of anti-retroviral drug treatment include nausea, vomiting, heart disease, diabetes and liver damage.

In view of the difficulties associated with anti-retroviral drug treatment there is an urgent need for greater understanding of the host-retrovirus interaction and to identify effective methods and reagents for controlling retroviral infections and improving current anti-retroviral drug treatment regimens particularly to facilitate their long term efficacy. Also, in view of the undesirable and often severe side-effects, there is a need for treatment protocols which allow periods in which anti-retroviral drugs are not administered. As a result of the onerous and intrusive treatment regimens, there is a demand from patients for protocols which allow them periods in which they do not take anti-retroviral drugs.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers 21 400>1 (SEQ ID NO:1), <400>2 (SEQ ID NO:2), etc. A summary of sequence identifiers is provided in Table 1. A sequence listing is provided after the claims.

The present invention is predicated, in part, on the development of a vector which expresses a retrovirus antigen and/or a host modulatory factor and which upon administration to a subject is capable of reducing, preventing or delaying viral rebound or of reducing, preventing or delaying the increase or rate of increase in viral load in a subject. As there are significant disadvantages and difficulties with present anti-retroviral drug treatment regimens in terms of their efficacy, side effects and compliance, it is anticipated that the vectors of the present invention will find broad application in the treatment of retroviral infections in conjunction with anti-retroviral drug treatment.

In one aspect, the present vector is a poxvirus vector which expresses one or more retrovirus antigens and/or a cytokine and is administered in conjunction with anti-retroviral drug therapy. In some embodiments a retrovirus antigen and a cytokine are co-expressed by the poxivirus vector.

In one embodiment, the invention provides a recombinant poxvirus vector comprising a sequence of nucleotides encoding a retrovirus antigen or a functional homolog, derivative, part or analog thereof, and a sequence of nucleotides encoding a cytokine or a functional homolog, derivative, part or analog thereof, for use in conjunction with anti-retroviral drug therapy to maintain or prolong a low retroviral load in a subject and to prevent, reduce or delay viral rebound during interruption of anti-retroviral drug treatment in a subject.

In some embodiments, the recombinant poxvirus vector comprises a sequence of nucleotides encoding a retrovirus antigen and a sequence of nucleotides encoding a cytokine, or a functional homolog, derivative, part or analog thereof, for use in conjunction with anti-retroviral drug therapy wherein the antigen and the cytokine are co-expressed and are effective in reducing or alleviating one or more of the side effects of anti-retroviral drug therapy.

In other embodiments, the vector comprises a sequence of nucleotides encoding a retrovirus antigen or a functional homolog, derivative, part or analog thereof, and a sequence of nucleotides encoding a cytokine or a functional homolog, derivative, part or analog thereof, for use in reducing or alleviating one or more side effects of anti-retroviral drug therapy.

In particular embodiments, the cytokine is selected from IFNγ, IL-12, IL-2, TNF and IL-6. In some embodiments the cytokine is IFNγ.

In certain embodiments the IFNγ comprises the amino acid sequence set forth in SEQ ID NO: 6 or an amino acid sequence having at least about 60% similarity thereto. In other embodiments IFNγ is encoded by a sequence of nucleotides set forth in SEQ ID NO: 5 or a sequence of nucleotides encoding a functional homolog or derivative thereof having at least 60% similarity thereto or a sequence which hybridises thereto or to a complementary form thereof under conditions of medium stringency.

In some embodiments, the retroviral antigen is encoded by a coding region selected from gag, env, pol and pro coding regions. In preferred embodiments, the retroviral antigen is antigen is encoded by gag and/or pol coding regions, wherein the gag and/or pol coding region of HV being particularly preferred. In particular embodiments, the retroviral antigens encoded by gag and pol comprise the amino acid sequence set forth in SEQ ID NO: 2 and SEQ ID NO:4, respectively or a functional homolog, part or derivative thereof comprising a sequence of amino acids having at least 60% similarity thereto. In other embodiments, the gag is encoded by a sequence of nucleotides set forth in SEQ ID NO: 1 or a sequence of nucleotides encoding a functional homolog, part or derivative thereof having at least 60% similarity thereto after optimal alignment or a sequence which hybridises thereto or to a complementary form thereof under conditions of medium stringency, and wherein pol is encoded by a sequence of nucleotides set forth in SEQ ID NO: 3 or a sequence of nucleotides encoding a functional homolog, part or derivative thereof having at least 60% similarity thereto after optimal alignment or a sequence which hybridises thereto or to a complementary form thereof under conditions of medium stringency. The present invention is exemplified with respect to fowlpox virus vectors but extends to the use of other avipox virus vectors.

In some aspects, the present invention provides a method for treatment or prophylaxis of one or more symptoms of a retrovirus infection such as HIV infection, comprising the administration of a poxvirus vector encoding a retrovirus antigen and a cytokine, or a functional homolog, derivative, part or analog thereof, in conjunction with anti-retroviral drug therapy wherein said polypeptide and/or cytokine are expressed in a subject and are effective in maintaining a low viral load in a subject for a period of time, for example effectively preventing, reducing or delaying viral rebound during interruption of anti-retroviral drug treatment.

In other embodiments the method comprises administering to a subject a poxvirus vector encoding an antigen of the retrovirus or the retrovirus antigen and a cytokine, or a functional homolog, derivative, part or analog of the retrovirus antigen and/or the cytokine, in conjunction with anti-retroviral drug therapy wherein the antigen or the antigen and the cytokine are expressed in the subject and are effective in maintaining or prolonging a low retroviral load in the subject for a period of time and are effective in preventing, reducing or delaying viral rebound during interruption of anti-retroviral drug treatment.

In another embodiment, the vector is administered to a subject exhibiting a low retroviral viral load as a result of anti-retroviral drug therapy. In other embodiments, the vector is administered to a subject exhibiting a low retroviral load prior to commencement of anti-retroviral drug therapy.

In particular embodiments, the cytokine is selected from IFNγ, IL-12, IL-2, TNF and IL-6. In some embodiments the cytokine is IFNγ.

In certain embodiments the IFNγ comprises the amino acid sequence set forth in SEQ ID NO: 6 or an amino acid sequence having at least about 60% similarity thereto. In other embodiments IFNγ is encoded by a sequence of nucleotides set forth in SEQ ID NO: 5 or a sequence of nucleotides encoding a functional homolog or derivative thereof having at least 60% similarity thereto or a sequence which hybridises thereto or to a complementary form thereof under conditions of medium stringency.

In some embodiments, the retroviral antigen is encoded by a coding region selected from gag, env, pol and pro coding regions. In preferred embodiments, the retroviral antigen is antigen is encoded by gag and/or pol coding regions, wherein the gag and/or pol coding region of HIV being particularly preferred. In particular embodiments, the retroviral antigent encoded by gag and pol comprise the amino acid sequence set forth in SEQ ID NO: 2 and SEQ ID NO:4, respectively or a functional homolog, part or derivative thereof comprising a sequence of amino acids having at least 60% similarity thereto. In other embodiments, the gag is encoded by a sequence of nucleotides set forth in SEQ ID NO: I or a sequence of nucleotides encoding a functional homolog, part or derivative thereof having at least 60% similarity thereto after optimal alignment or a sequence which hybridises thereto or to a complementary form thereof under conditions of medium stringency, and wherein pol is encoded by a sequence of nucleotides set forth in SEQ ID NO: 3 or a sequence of nucleotides encoding a functional homolog, part or derivative thereof having at least 60% similarity thereto after optimal alignment or a sequence which hybridises thereto or to a complementary form thereof under conditions of medium stringency. The present invention is exemplified with respect to fowlpox virus vectors but extends to the use of other avipox virus vectors.

In other embodiment, the present invention provides a method for the treatment or prophylaxis of HIV/AIDS comprising administering to a subject a poxvirus vector comprising a sequence of nucleotides encoding a retrovirus antigen and a sequence of nucleotides encoding a cytokine, or a functional homolog, part, derivative or analogue thereof in conjunction with anti-retroviral drug therapy, wherein said method is effective in maintaining a low retroviral load in the subject or preventing, reducing or delaying viral rebound in the absence of anti-retroviral drug therapy.

In another embodiment, the method comprises administering to a subject a poxvirus vector comprising a sequence of nucleotides encoding a retrovirus antigen and a sequence of nucleotides encoding a cytokine, or a functional homolog, part, derivative or analog of the antigen and/or the cytokine, in conjunction with anti-retroviral drug therapy, wherein said method is effective in maintaining a low retroviral load in the subject and preventing, reducing or delaying retroviral rebound in the absence of anti-retroviral drug therapy.

In some embodiments, the retrovirus antigen is an HIV antigen.

Methods are also provided in one embodiment, for reducing or alleviating one or more side effects of ARDT comprising administering the instant vectors for a time and under conditions to reduce or alleviate one or more of the said effects of ARDT. The vectors may be administered before and/or during ARDT and/or after withdrawal of ARDT. The present method facilitate inter alia a treatment program involving interrupted ARDT with a concomitant reduction or alleviation of its side effects.

In this embodiment, the method comprises administering to a subject exhibiting a retroviral infection a poxvirus vector comprising a sequence of nucleotides encoding an antigen of the retrovirus or a functional derivative, homolog, part or analog thereof, and a sequence of nucleotides encoding a cytokine or a functional derivative, homolog, part or analog thereof, for a time and under conditions sufficient to co-express the antigen and the cytokine and to reduce or alleviate one or more side effects of anti-retroviral drug therapy in the subject.

Particularly, the present invention provides a method of reducing or alleviating one or more side effects of anti-retroviral drug therapy comprising administering to a subject a poxvirus vector comprising a sequence of nucleotides encoding a retrovirus antigen and a sequence of nucleotides encoding a cytokine, or a functional derivative, homolog, part or analog thereof, for a time and under conditions sufficient to co-express the antigen and the cytokine and to reduce or alleviate one or more side effects of anti-retroviral drug therapy in the subject.

In some embodiments, the vector is administered to a subject exhibiting a low retroviral viral load as a result of anti-retroviral drug therapy. In other embodiments, the vector is administered to a subject exhibiting a low retroviral load prior to commencement of anti-retroviral drug therapy.

In particular embodiments, the cytokine is selected from IFNγ, IL-12, IL-2, INF and IL-6. In some embodiments the cytokine is IFNγ.

In certain embodiments the IFNγ comprises the amino acid sequence set forth in SEQ ID NO: 6 or an amino acid sequence having at least about 60% similarity thereto. In other embodiments IFNγ is encoded by a sequence of nucleotides set forth in SEQ ID NO: 5 or a sequence of nucleotides encoding a functional homolog or derivative thereof having at least 60% similarity thereto or a sequence which hybridises thereto or to a complementary form thereof under conditions of medium stringency.

In some embodiments, the retroviral antigen is encoded by a coding region selected from gag, env, pol and pro coding regions. In preferred embodiments, the retroviral antigen is antigen is encoded by gag and/or pol coding regions, wherein the gag and/or pol coding region of HIV being particularly preferred. In particular embodiments, the retroviral antigent encoded by gag and pol comprise the amino acid sequence set forth in SEQ ID NO: 2 and SEQ ID NO:4, respectively or a functional homolog, part or derivative thereof comprising a sequence of amino acids having at least 60% similarity thereto. In other embodiments, the gag is encoded by a sequence of nucleotides set forth in SEQ ID NO: 1 or a sequence of nucleotides encoding a functional homolog, part or derivative thereof having at least 60% similarity thereto after optimal alignment or a sequence which hybridises thereto or to a complementary form thereof under conditions of medium stringency, and wherein pol is encoded by a sequence of nucleotides set forth in SEQ ID NO: 3 or a sequence of nucleotides encoding a functional homolog, part or derivative thereof having at least 60% similarity thereto after optimal alignment or a sequence which hybridises thereto or to a complementary form thereof under conditions of medium stringency. The present invention is exemplified with respect to fowlpox virus vectors but extends to the use of other avipox virus vectors.

In a related aspect, the present invention contemplates a use of a recombinant vector comprising a sequence of nucleotides encoding a retrovirus antigen and a sequence of nucleotides encoding a cytokine, or a functional derivative, homolog, part or analog thereof in the manufacture of a medicament for use in the treatment or prophylaxis of one or more symptoms of a retroviral infection such as HIV infection wherein the antigen and the cytokine are co-expressed in a subject and are effective in maintaining or prolonging a low retroviral load in the subject for a period of time and/or are effective in preventing, reducing or delaying viral rebound during interruption of anti-retroviral drug treatment.

The method also contemplates in some embodiments, a use of a recombinant vector comprising a sequence of nucleotides encoding a retrovirus antigen or a functional derivative, homolog, part or analog thereof, and a sequence of nucleotides encoding a cytokine or a functional derivative, homolog, part or analog thereof in the manufacture of a medicament for use in maintaining or prolonging a low retroviral load in a subject for a period of time, and in preventing, reducing or delaying viral rebound during interruption of anti-retroviral drug treatment.

In some embodiments, the invention provides a use of a recombinant vector comprising a sequence of nucleotides encoding a retrovirus antigen and/or a sequence of nucleotides encoding a cytokine, or a functional derivative, homolog, part or analog thereof in the manufacture of a medicament for use in reducing or alleviating one or more of the side effects of anti-retroviral drug therapy.

In an exemplary embodiment, the vector is a fowlpox vector co-expressing gag/pol and IFNγ which effectively maintains a low viral vector load, or delays the increase in viral load during a period when antiretroviral drug treatment is interrupted. The present invention extends to pharmaceutical agents comprising the vectors of the present invention and their use in a range of treatment regimens.

### A summary of sequence identifiers used throughout the subject specification is provided in Table 1.

TABLE 1
Summary of sequence identifiers

| SEQUENCE | |
|---|---|
| ID NO: | DESCRIPTION |
| 1 | Nucleotide sequence encoding HIV gag |
| 2 | Amino acid sequence encoded by SEQ ID NO: 1 |
| 3 | Nucleotide sequence encoding HIV pol |
| 4 | Amino acid sequence encoded by SEQ ID NO: 3 |
| 5 | Nucleotide sequence encoding human IFNγ |
| 6 | Amino acid sequence encoded by SEQ ID NO: 5 |
| 7 | Nucleotide sequence of insertion site of rFPV gag/pol |
| | IFNγ |

### BRIEF DESCRIPTION OF THE FIGURES

FIG. 1 is a graphical representation showing the mean viral load (non-log) over the 20 week period of the extension trial for each vector recipient group. Subject Group A (white line) received the full construct (FC) comprising recombinant FPV expressing HIV-1 gag/pol and interferon-gamma (IFNγ). Subject Group B (black line) received the partial construct (PC) comprising recombinant FPV expressing HIV-1 gag/pol. Subject Group C (grey line) received diluent alone (placebo).

FIG. 2 is a graphical representation showing the proportion of recipients in each recipient group whose viral load was low enough over the period of the study (in days) such that ARDT was not re-initiated. Subject Group A received the full construct (FC) comprising recombinant FPV expressing HIV-1 gag/pol and interferon-gamma (IFNγ). Subject Group B received the partial construct (PC) comprising recombinant FPV expressing HIV-1 gag/pol. Subject Group C received diluent alone (placebo).

FIG. 3 is an annotated representation of the nucleotide sequence (SEQ ID NO: 7) of the insertion site of the recombinant FPV gag/pol IFNγ employed in the Examples.

### DETAILED DESCRIPTION OF THE INVENTION

The present invention provides a vector which effectively modulates retroviral load in a subject. Specifically, the vector of the present invention maintains or prolongs a low viral load in a subject infected with a retroviral infection. In a preferred aspect the vector of the present invention is used in conjunction with anti-retroviral drug therapy and is useful in maintaining a low viral load before, after or between periods of drug therapy.

In one aspect, the present invention provides a recombinant vector comprising a sequence of nucleotides encoding a retrovirus antigen and/or a sequence of nucleotides encoding a modulatory factor, or a functional homolog, derivative, part or analog thereof, which expresses said sequences for use in conjunction with ARDT in the treatment or prophylaxis of one or more symptoms associated with a retroviral infection in a subject.

In some embodiments, the present invention provides a recombinant vector comprising a sequence of nucleotides encoding a retrovirus antigen and/or a sequence of nucleotides encoding a modulatory factor, or a functional homolog, derivative, part or analog thereof, which expresses said sequences when used in conjunction with ARDT in the treatment or prophylaxis of one or more symptoms associated with a retroviral infection in a subject.

Reference herein to anti-retroviral drug treatment (ARDT) is used in its broadest context to include the use of one or more compounds, singly or in combination in regimens for retroviral, and in particular HIV retroviral treatment.

Anti-retroviral compounds act by a number of different of mechanisms which selectively affect the virus. For example, protease inhibitors, reverse transcriptase inhibitors and ribonucleotide reduction inhibitors may be employed or compounds which inhibit viral adsorption, assembly, integration and transcription. As will be known to those skilled in the art there are a large number of anti-retroviral compounds which may be administered. Examples of protease inhibitors include Indinavir and Nelfinavir. Reverse transcriptase inhibitors include, for example, Zidovisdine, Stavudine and Didanosine. Examples of ribonucleotide reductase inhibitors include thiosemicarbazone derivatives.

The particular compounds and combinations used and the dosages and regimens will be determined by the administering practitioner and will depend inter alia, upon individual responses to the treatment.

In another aspect, the present invention provides a recombinant vector comprising a sequence of nucleotides encoding a retrovirus antigen and a sequence of nucleotides encoding a modulatory factor, or a functional homolog, derivative, part or analog thereof, which co-expresses said constituents for use in conjunction with ARDT in the treatment or prophylaxis of one or more symptoms associated with a retroviral infection in a subject.

As used herein the singular forms “a”, “an” and “the” include plural aspects unless the context clearly dictates otherwise. Thus, for example, reference to a “compound” includes a single compound, as well as two or more compounds; reference to “an active agent” includes a single active agent, as well as two or more active agents; and so forth.

The term “antigen” is used in its broadest context to include molecules comprising one or more epitopes against which an immune response is produced. The term however, also includes within its scope any polypeptide, including a protein or peptide. Antigenic portions may be identified using well known techniques, such as those set out in Paul, Fundamental Immunology, 3^{rd} Ed., 243-247 (Raven Press, 1993) and references cited therein.

The term “recombinant vector” is used herein in its broadest sense as a reference to constructs which are capable of vectoring or carrying nucleic acid molecules into a target cell for expression therein. The vectors of the present invention include viral vectors or similar constructs or derivatives thereof, plasmid vectors or naked nucleic acid molecules.

Poxvirus vectors are particularly convenient vectors. As used herein reference to “poxvirus” includes viruses selected from the group comprising avipox (eg, fowlpox, canarypox, pigeonpox) orthopox (eg, vaccinia) capripox (eg, sheep, goats) and suipox (eg, swinepox). Preferred poxvirus vectors are avipox or orthopox vectors. Avipox vectors are preferred vectors. A particularly preferred avipoxvirus vector is a fowlpox vector (FPV). Exemplary fowlpox vectors are FPV-M3 vectors as described in International Patent Publication No. WO 00/28003. The principles and procedures for generating and using recombinant poxvirus vectors are well known in the art. Briefly, homologous recombination between a donor recombination vector and a poxvirus within a host cell permits correct introduction of the desired sequences.

Reference to “modulates” includes down regulation of viral load, maintenance of viral load and a change in the rate of increase of viral load. Specifically, any change in viral load is usually but not exclusively determined over an appropriate period of time and is expressed in terms of change in average viral load over time of a subject or group of subjects.

Accordingly, the present invention provides a recombinant poxvirus vector comprising a sequence of nucleotides encoding a retrovirus antigen and a sequence of nucleotides encoding a modulatory factor, or a functional homolog, derivative, part or analog thereof, which co-expresses said constituents for use in conjunction with ARDT in the treatment or prophylaxis of one or more symptoms associated with a retroviral infection in a subject.

Reference to “treatment” and “prophylaxis” are to be considered in their broadest context. The term treatment includes partial and full recovery of HIV infection or of the clinical symptoms of AIDS. The term “prophylaxis” includes a delay in contracting an HIV infection or experiencing symptoms of HIV infection including the clinical symptoms of AIDS. Certain symptoms are shared between symptoms of an HIV infection, and the clinical symptoms of AIDS. As will be understood by one skilled in the art, examples of shared symptoms include a detectable viral load and reduced levels of CD4+ cells. Certain HIV infected individuals have a low viral load and fail to show the clinical symptoms of AIDS such as immunosuppression, wasting diseases or increased levels of opportunistic infections. Accordingly, the vectors of the present invention are used to treat the symptoms of HIV infection and clinical symptoms of AIDS and AIDS related disorders.

Although human subjects are primarily contemplated, reference to a “subject” should be understood to include mammals including primates (eg, humans, monkeys), livestock animals (eg, sheep, cows, horses, donkeys, goats, pigs), laboratory test animals (eg, mice, rats, ducks, dogs, guinea pigs, rabbits, hampsters), companion animals (eg, dogs, cats, birds), and captive wild animals (eg, kangaroos, deer, foxes). Preferably said subject is a mammal, more preferably a primate and even more preferably a human.

The phrase “modulatory factor” is used herein include to those host factors which act as chemical messengers between cells to effect change in response to external or internal stimuli. Thus ligands for cellular receptors such as cytokines, growth factors and chemokines are contemplated together with their functional homologs, parts, derivatives and analogs. As will be understood by those skilled in the art, the activity of such a factor may also be achieved through the administration of a compound which acts as an agonist of said factor or as an antagonist of inhibitors of the said factor or by down stream effectors in the same pathway or network. Accordingly, the term modulatory factors includes reference to the host factor, its down stream effectors and agonists thereof.

In a further embodiment, the present invention provides a recombinant vector comprising a sequence of nucleotides encoding a retrovirus antigen and a sequence of nucleotides encoding a cytokine, or a functional homolog, derivative, part or analog thereof, which co-expresses said sequences for use in conjunction with ARDT in the treatment or prophylaxis of one or more symptoms associated with a retroviral infection in a subject.

In a further embodiment, the present invention provides a recombinant poxvirus vector comprising a sequence of nucleotides encoding a retrovirus antigen and a sequence of nucleotides encoding a cytokine, or a functional homolog, derivative, part or analog thereof, which co-expresses said sequences for use in conjunction with ARDT in the treatment or prophylaxis of one or more symptoms associated with a retroviral infection in a subject.

In a particularly preferred embodiment, the modulatory factor of the present invention is selected from IFNγ, IL-12, IL-2, INF and IL-6 and down stream effectors and agonists thereof. IFNγ is exemplified herein and IFNγ or its functional homologs, parts, derivatives and analogs are preferred.

In a preferred, aspect the present invention provides a recombinant poxvirus vector comprising a sequence of nucleotides encoding a retrovirus antigen and a sequence of nucleotides encoding IFNγ, or a functional homolog, derivative, part or analog thereof, which co-expresses said constituents for use in conjunction with ARDT in the treatment or prophylaxis of one or more symptoms of a retroviral infection in a subject.

Preferred retroviral antigens include those encoded by a coding regions selected from gag, env, pol and pro coding regions.

Particularly preferred antigens are those encoded by gag and/or pol coding regions. A gag/pol construct is also preferred.

The present invention is particularly directed to the treatment of human retroviral infections such as HIV and preferably HIV-1.

In a particularly preferred embodiment the retroviral antigens are encoded by gag and pol coding regions derived from HIV and preferably HIV-1.

Accordingly, in another preferred aspect the present invention provides a recombinant poxvirus vector comprising a sequence of nucleotides encoding gag and/or pol antigens from HIV and a sequence of nucleotides encoding IFNγ, or a functional homologue, derivative, part, or analogue thereof, which vector co-expresses said sequences for use in conjunction with ARDT in the treatment or prophylaxis of one or more symptoms of an HIV infection or AIDS in a subject.

Accordingly, in another aspect the present invention provides a recombinant poxvirus vector comprising a sequence of nucleotides encoding gag and pol antigens from HIV and a sequence of nucleotides encoding IFNγ, or a functional homologue, derivative, part, or analogue thereof, which vector co-expresses said sequences for use in conjunction with ARDT in the treatment or prophylaxis of one or more symptoms of an HIV infection or AIDS in a subject.

Preferably said poxvirus is a fowlpox virus.

In a further embodiment, the gag antigen is encoded by a sequence of nucleotides set forth in SEQ ID NO: 1 or a sequence of nucleotides having at least 60% similarity thereto after optimal alignment or a sequence which hybridises thereto or to a complementary form thereof under conditions of medium stringency.

In a further embodiment, the gag antigen is comprises a sequence of amino acids set forth in SEQ ID NO: 2 or a sequence of amino acids having at least 60% similarity thereto after optimal alignment.

In a further embodiment, the pol antigen is encoded by a sequence of nucleotides set forth in SEQ ID NO: 3 or a sequence of nucleotides having at least 60% similarity thereto after optimal alignment or a sequence which hybridises thereto or to a complementary form thereof under conditions of medium stringency.

In a further embodiment, the pol antigen is comprises a sequence of amino acids set forth in SEQ ID NO: 4 or a sequence of amino acids having at least 60% similarity thereto after optimal alignment.

In a further embodiment, the IFNγ antigen is encoded by a sequence of nucleotides set forth in SEQ ID NO: 5 or a sequence of nucleotides having at least 60% similarity thereto or a sequence which hybridises thereto or to a complementary form thereof under conditions of medium stringency.

In a further embodiment, the IFNγ antigen is comprises a sequence of amino acids set forth in SEQ ID NO: 6 or a sequence of amino acids having at least 60% similarity thereto after optimal alignment.

In a further embodiment, the vector is fowlpox vector comprising the nucleotide sequence set forth in SEQ ID NO: 7 or a sequence of nucleotides having at least 60% similarity thereto or a sequence which hybridises thereto or to a complementary form thereof under conditions of medium stringency.

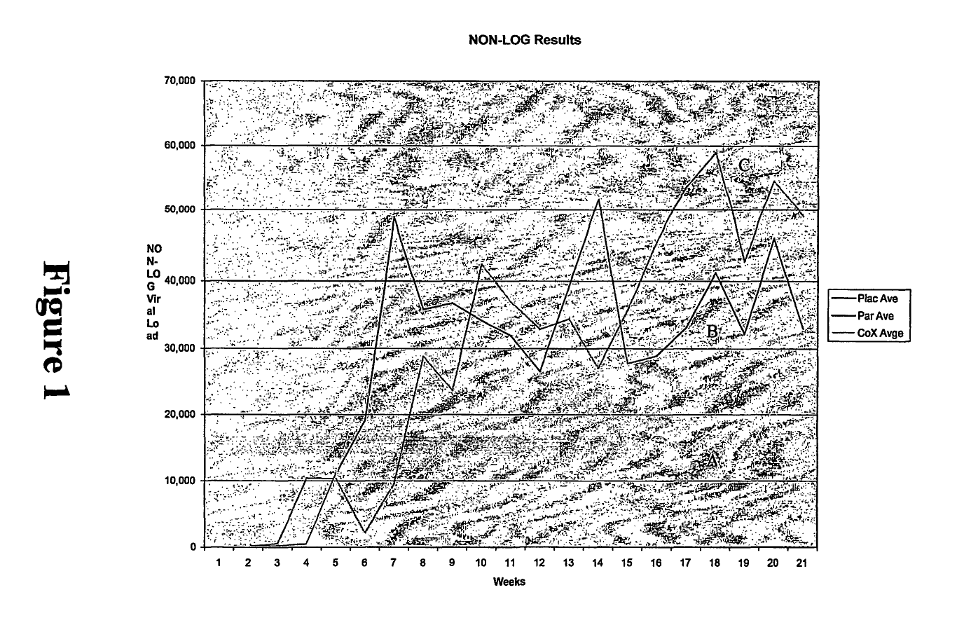

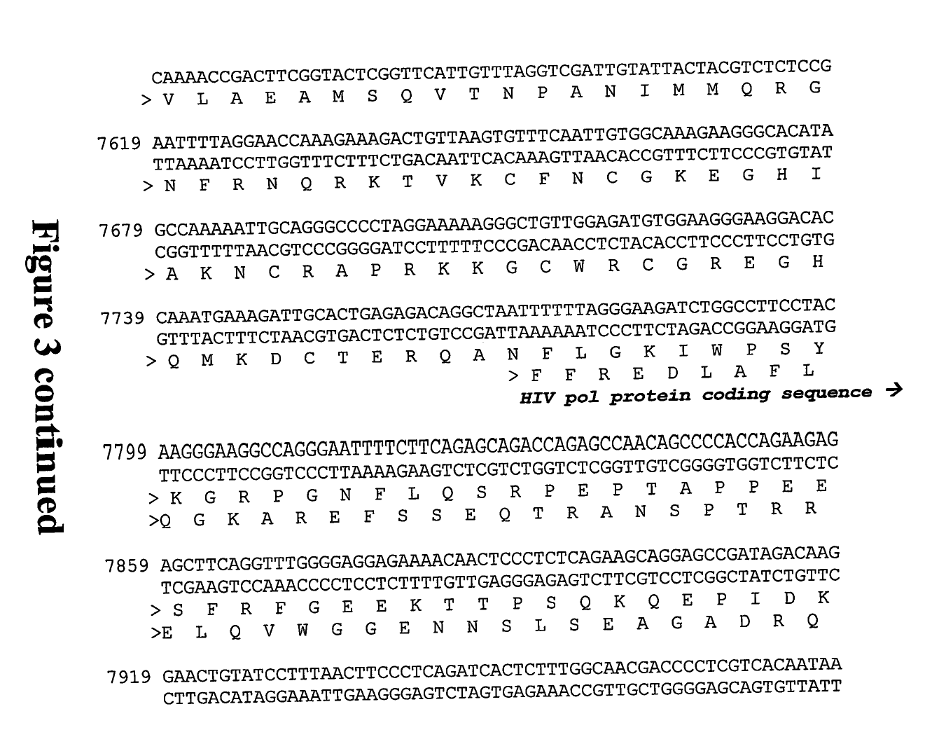

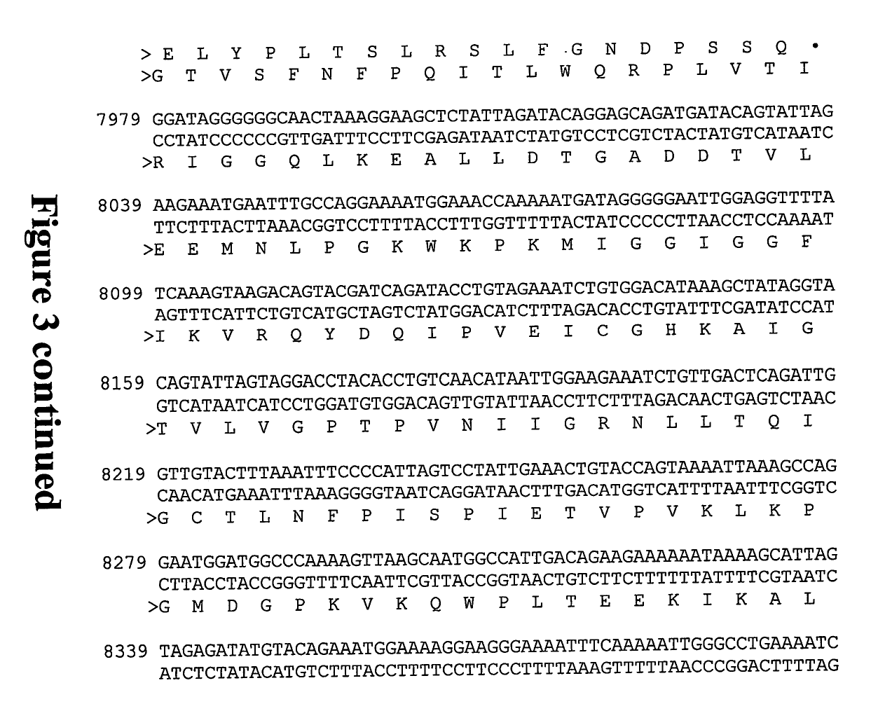

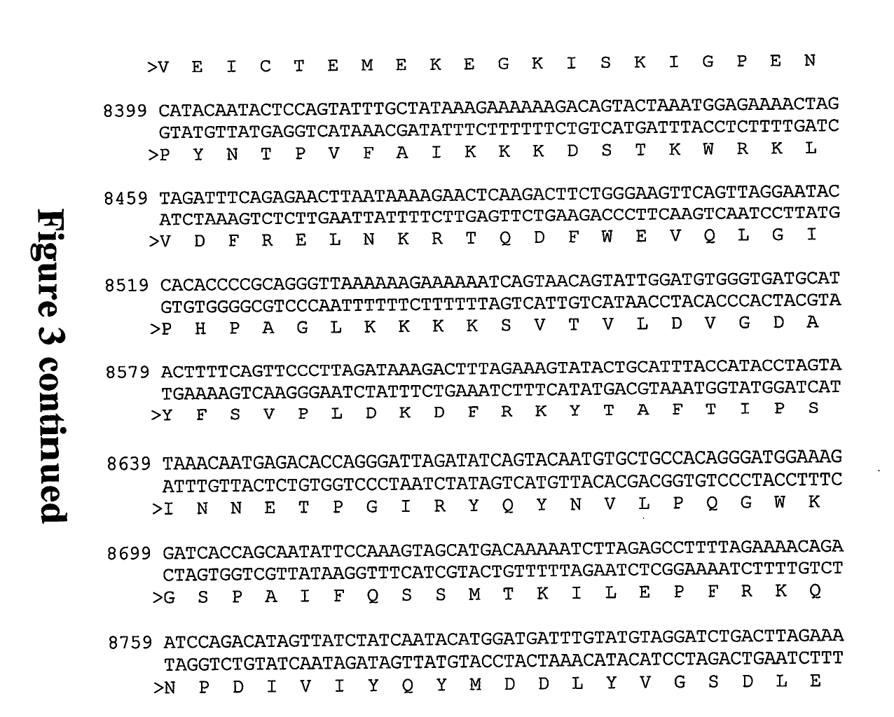

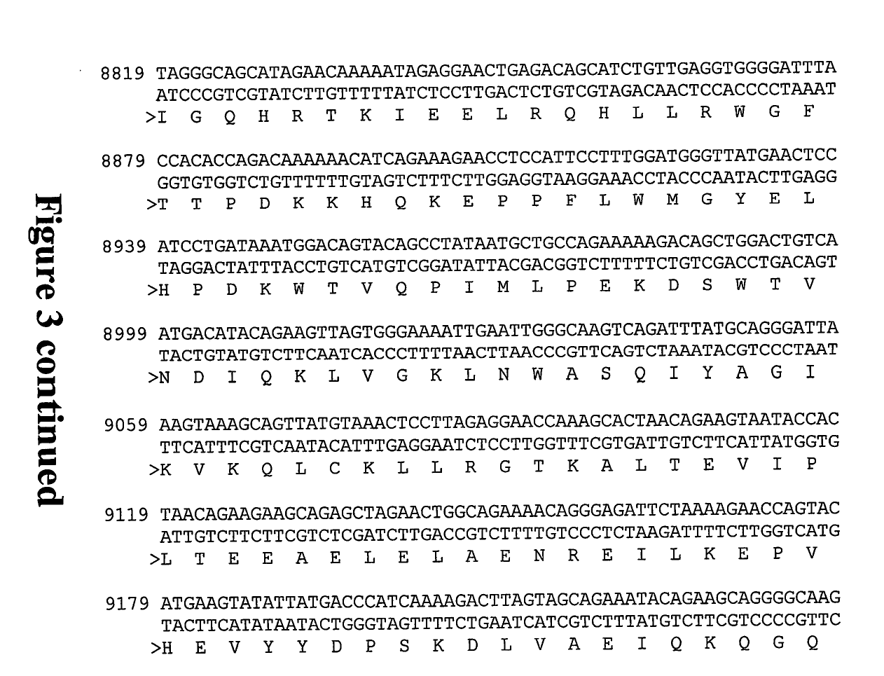

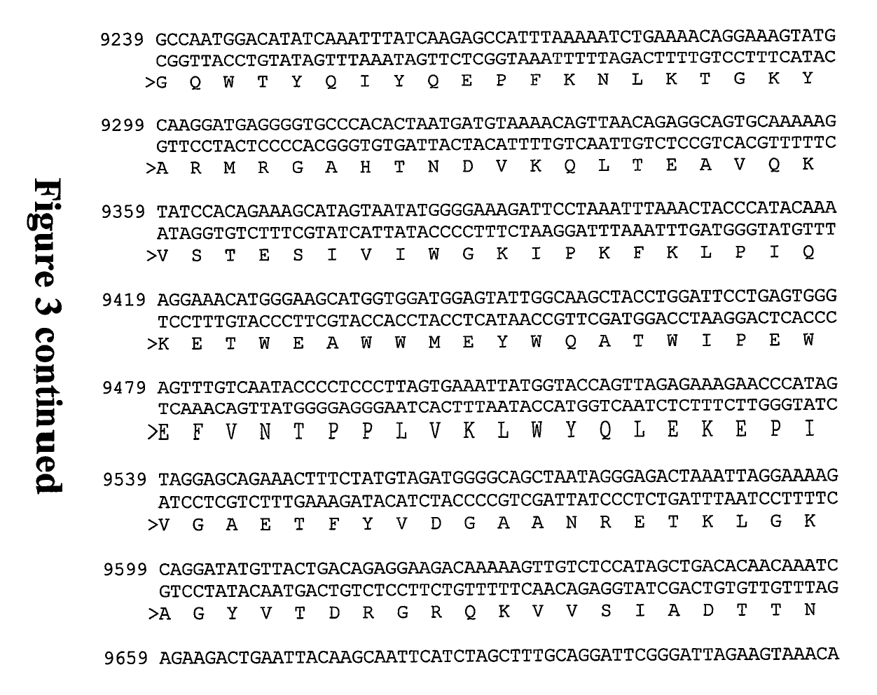

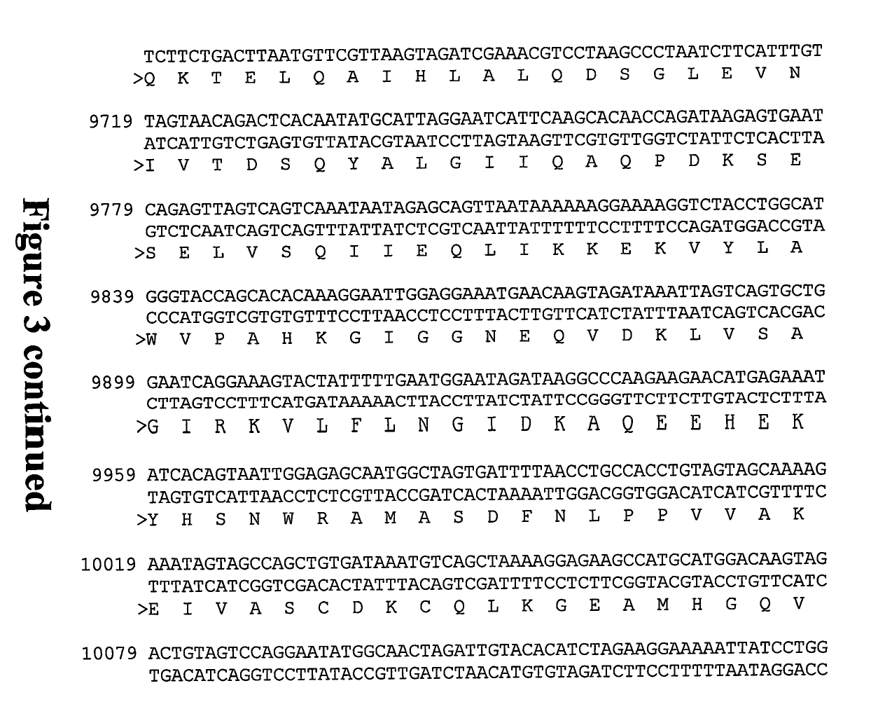

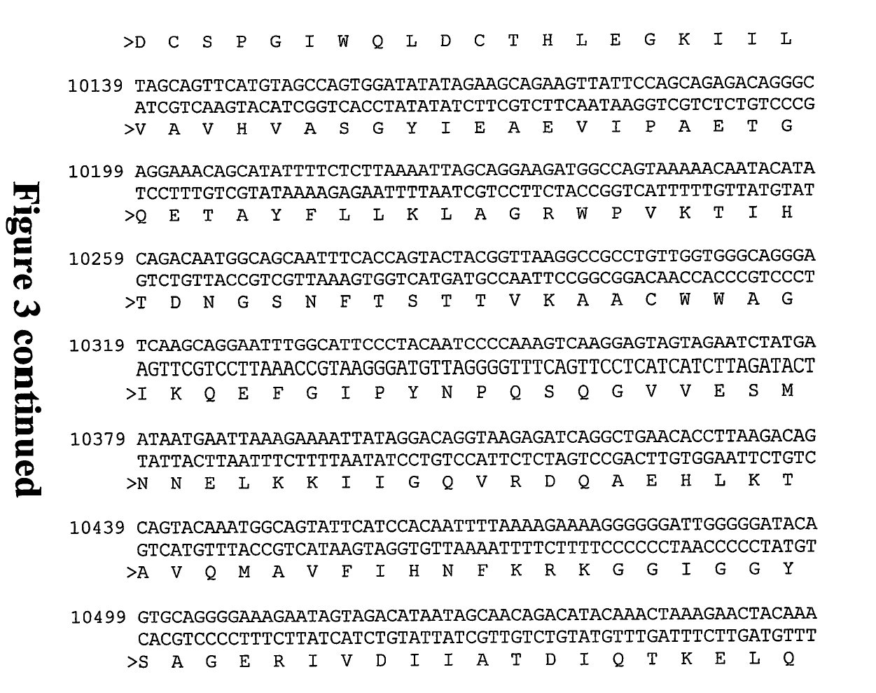

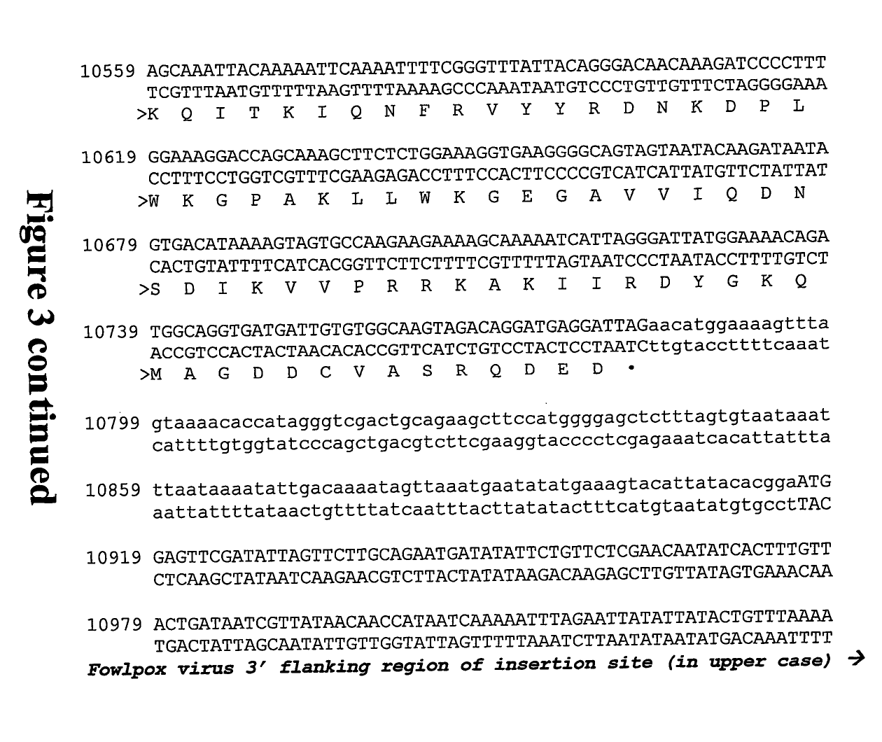

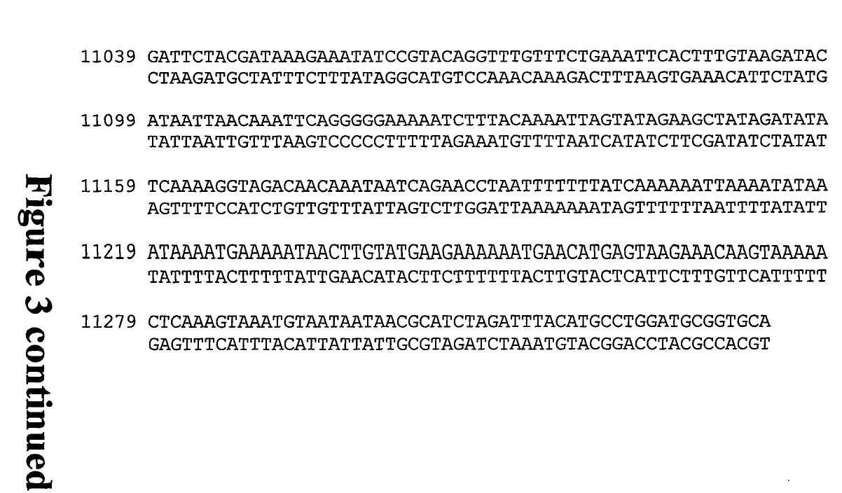

SEQ ID NO: 7 and FIG. 3 provide the sequence of the insertion site of rFPV gag/pol IFNγ representing the exemplified embodiments. As shown in FIG. 1, subjects administered with this construct showed an approximately 10 fold reduction in average viral load over the period of the study and in the absence of ARDT. This resulted in at least a delay in the re-initiation of ARDT for most subjects.

A “functional homolog” include species homologs whose function is conserved between species. Thus a functional homology of IFNγ retains its modulatory function. A functional homolog of pol, for example, retains its antigenic or biochemical function.

A “functional derivative” of an antigen or modulatory factor encompasses variants and portions or a part of a full length polypeptide, which retains the functional activity of the parent molecule. Such, active fragments include deletion mutants and small peptides, for example, of at least 10, preferably at least 20 and more preferably at least 30 contiguous amino acids, which exhibit the requisite activity. Peptides of this type may be obtained through the application of standard recombinant nucleic acid techniques or synthesized using conventional liquid or solid phase synthesis as described in Chapter 9 entitled “Peptide Synthesis” by Atherton and Shephard which is included in a publication entitled “Synthetic Vaccines” edited by Nicholson and published by Blackwell Scientific Publications.

The term “functional” means that the molecules retain or exceed the overall function of the parent. Accordingly, if in particular function is diminished in the derivative or homolog, this is compensated for new functions such as, for example, greater antigenicity, longevity, half-life, activity, avidity etc.

The term “variant” refers to nucleotide sequences displaying substantial sequence identity with a reference nucleotide sequences or polynucleotides that hybridize with a reference sequence under stringency conditions that are defined hereinafter. The terms “nucleotide sequence”, “polynucleotide” and “nucleic acid molecule” may be used herein interchangeably and encompass polynucleotides in which one or more nucleotides have been added or deleted, or replaced with different nucleotides. In this regard, it is well understood in the art that certain alterations inclusive of mutations, additions, deletions and substitutions can be made to a reference nucleotide sequence whereby the altered polynucleotide retains the biological function or activity of the reference polynucleotide. The term “variant” also includes naturally-occurring allelic variants.

Functional derivatives of a target molecule include active portions of the target molecule whose modification in a subject ameliorates a disease or condition and which may be further modified to enhance this affect. A functional derivative of a target molecule in the form of a protein or peptide comprises a sequence of amino acids having at least 60% similarity to the target molecule or portion thereof. A “portion” in peptide form may be as small as an epitope comprising less than 5 amino acids or as large as several hundred kilodaltons. The length of the polypeptide sequences compared for homology will generally be at least about 16 amino acids, usually at least about 20 residues, more usually at least about 24 residues, typically at least about 28 residues and preferably more than about 35 residues.

When in nucleic acid form, a functional derivative comprises a sequence of nucleotides having at least 60% similarity to the target molecule after optimal alignment. A “portion” of a target nucleic acid molecule is defined as having a minimal size of at least about 10 nucleotides or preferably about 13 nucleotides or more preferably at least about 20 nucleotides and may have a minimal size of at least about 35 nucleotides. This definition includes all sizes in the range of 10-35 nucleotides including 10, 11, 12, 13, 14, 15, 16, 17, 18, 19, 20, 21, 22, 23, 24, 25, 26, 27, 28, 29, 30, 31, 32, 33, 34 or 35 nucleotides as well as greater than 35 nucleotides including 50, 100, 300, 500, 600 nucleotides or nucleic acid molecules having any number of nucleotides within these values.

Functional derivatives of target molecules in nucleic acid form include nucleic acid molecules comprising a nucleotide sequence capable of hybridising to the target molecule or its complementary form under low stringency conditions.

Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (INBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyro carbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N _{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

These types of molecules may be important to stabilise vector constructs or their expressed products.

The terms “similarity” or “identity” as used herein include exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Preferably, the percentage similarity between a particular sequence and a reference sequence (nucleotide or amino acid) is at least about 60% or at least about 70% or at least about 80% or at least about 90% or at least about 95% or above such as at least about 96%, 97%, 98%, 99% or greater. Percentage similarities or identities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 2542° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C. High stringency conditions are particularly preferred.

The present invention contemplates expression of the nucleotide sequences encoding the modulatory factor and/or the retroviral antigen in recipient cells. However, appropriate alternative means to deliver said agents to recipient cells may be practiced within the scope of the present invention. Thus, the modulatory factor may be administered in proteinaceous or other suitable and pharmaceutically acceptable chemical form optionally in conjunction with the vector of the present invention comprising a nucleotide sequence encoding a retroviral antigen and/or said modulatory factor.

In another aspect the present invention provides a pharmaceutical composition comprising any one of the above-described vectors together with a pharmaceutically acceptable carrier and/or diluent for use in conjunction with ARDT in the treatment or prevention of a retroviral infection.

The term pharmaceutical composition is used herein to refer to a chemical compound which induces a desired pharmacological and/or physiological effect. The term encompasses pharmaceutically acceptable and pharmacologically active ingredients of the active agent and includes pharmaceutically acceptable and pharmacologically active salts, esters, amides, pro-forms, metabolites, analogues, etc. The term “compound” as used herein is not to be construed as a chemical molecule only but extends to peptides, polypeptides, and proteins as well as nucleic acid molecules and chemical analogues thereof.

By “pharmaceutically acceptable” excipient or diluent is meant a pharmaceutical vehicle comprised of material which is not biologically or otherwise undesirable, ie the material may be administered without causing any or a substantial adverse reaction. Carriers may include excipients and other additives such as diluents, colouring agents, wetting or emulsifying agents, buffering agents, preservatives, and the like.

In a preferred aspect said pharmaceutical composition is useful in conjunction with anti-retroviral drug treatment to modulate viral load in a subject.

In another aspect, the present invention contemplates a recombinant vector comprising a sequence of nucleotides encoding a retroviral antigen and/or a sequence of nucleotides encoding a cytokine or a functional homolog, part, derivative or analogue thereof, wherein upon administration to a subject carrying a low retroviral load, said antigen and/or cytokine is expressed in target cells and said low viral load is effectively maintained or prolonged.

In another aspect, the present invention contemplates a recombinant vector comprising a sequence of nucleotides encoding a retroviral antigen and a sequence of nucleotides encoding a cytokine or a functional homolog, part, derivative or analog thereof, wherein upon administration to a subject carrying a low retroviral load, said nucleotide sequences are expressed in target cells and said low viral load is effectively maintained or prolonged.

Expression as used herein broadly is a reference to the production of a polypeptide from a nucleic acid molecule.

Viral load is measured in terms of the number of viral particles/ml of plasma and is a useful and direct measure of viral infection and a surrogate marker of efficacy in retroviral treatment regimens including drug treatments and immunisation protocols. In particular, anti-retroviral drug treatment is usually started in a patient when their viral load goes above or is maintained above about 50 viral particles/ml of plasma for an appropriate period of time. One of the consequences of stopping or interrupting anti-retroviral drug treatment is that the viral load may “rebound” to a level which is as high or higher than the level before treatment commenced. Such viral rebound when left untreated is associated with the progression in a subject to development of the symptoms of primary HIV infection or the clinical symptoms of AIDS, or a worsening thereof. Accordingly, another useful measure of the efficacy of a treatment regimen in a subject is the time to development of detectable plasma viral loads or the time to re-initiation of anti-retroviral drug treatment. As absolute viral numbers as well as relative numbers are diagnostic it is also useful to consider the maximum viral load in a subject as well as the time-weighted change from a baseline value over a treatment period or during a post- or inter-treatment period. The protocols used to measure and quantify plasma viral loads are well known in the art and typically employ RT-PCR.

Another measure of treatment success or clinical progression is the ratio of CD4:CD8 T-cells in a subject. Furthermore, the success of immunization strategies and a measure of the immune status of a subject may be gauged by measuring CD8 T-cell responses and/or antibody responses to specific antigens. Methods of determining the cellular, virological and immunological status of a subject are well known in the art and are, for example, described in Harlow and Lane, Antibodies: A Laboratory Manual, Cold Spring Harbor Laboratory, 1998, and references cited therein.

A low viral load is an average figure and is preferably less than an average over time of about 50,000 copies/ml plasma. Preferably the average low viral load is less than about 40,000 copies/ml, more preferably less than about 30,000 copies/ml, still more preferably less than about 20,000 copies/ml, still more preferably less than about 10,000 copies/ml, even still more preferably less than about 1000 copies/ml, or any number between these aforementioned figures or between 1000 and 0 or undetectable copies/ml such as between 1000 and 100 copies/ml, between 500 and 50 copies/ml, or between 750 and 80 copies/ml, etc. Most preferably a low viral load is below 50 copies/ml.

The delay in viral rebound or a delay in an increase in viral load is any time frame which is likely to convey clinical benefit and may be measured in days, weeks, months or years. As exemplified herein, the average maximum viral load for subjects receiving the full construct (FC) was about 20,000 copies/ml and this was monitored over the 20 weeks of the study during withdrawal from anti-retroviral therapy.

Poxvirus vectors are particularly convenient vectors. Preferred poxvirus vectors are avipox or orthopox vectors which do not replicate efficiently in human subjects. A particularly preferred poxvirus vector is a fowlpox vector (FPV). Exemplary fowlpox vectors are FPV-M3 vectors as described in International Patent Publication No. WO 00/28003.

In a particularly preferred embodiment, the cytokine is selected from IFNγ, IL-12, IL-2, TNF and IL-6 and down stream effectors and agonists thereof. IFNγ is exemplified herein and IFNγ or its functional homologs, parts, derivatives and analogs are preferred.

Preferred retroviral antigens include those encoded by a coding regions selected from gag, env, pol and pro coding regions.

Particularly preferred antigens are those encoded by gag and/or pol coding regions. A gag/pol construct is also preferred.

The preferred retrovirus is HIV-1.

In a further embodiment, the recombinant vector of the present invention is administered in conjunction with ARDT. By “in conjunction” is meant that the instant vector and ARDT are used together but not necessarily simultaneously in order to improve treatment efficacy. In accordance with the present invention treatment efficacy is improved by providing an alternative or additional treatment to ARDT wherein the deleterious side effects of ARDT are reduced. Specifically, administration of the instant vector permits a treatment protocol to be conducted in which anti-retroviral drugs may be taken intermittently,

Accordingly, the present invention provides a recombinant vector comprising a sequence of nucleotides encoding a retroviral antigen and a sequence of nucleotides encoding a cytokine or a functional homolog, part, derivative or analogue thereof, wherein upon administration to a subject carrying a low retroviral load as a result of ARDT, said antigens are expressed in target cells and said low viral load is effectively maintained or prolonged after or while ARDT is withdrawn.

For the avoidance of doubt, the instant vector may be administered before, during, after or between ARDT/s.

In a preferred aspect, the present invention provides a method of treatment or prophylaxis comprising the administration of a vector comprising a sequence of nucleotides encoding a retroviral antigen and/or a sequence of nucleotides encoding a cytokine or a functional homolog, part, derivative or analogue thereof in conjunction with ARDT wherein said method is effective in maintaining a low retroviral load in a subject or reducing or delaying viral rebound in said subject.

In a preferred aspect, administration of the instant vector effectively prevents or treats one or more of the symptoms of HIV infection or AIDS.

In another aspect the present invention provides a method of treatment or prophylaxis comprising the administration of a vector comprising a sequence of nucleotides encoding a retroviral antigen and a sequence of nucleotides encoding a cytokine or a functional homolog, part, derivative or analog thereof in conjunction with ARDT wherein said method is effective in maintaining a low retroviral load is a subject or reducing or delaying viral rebound in a subject.

In a preferred aspect administration of the instant vectors effectively prevents or treats one or more of the symptoms of HIV infection or AIDS.

In a related aspect, the present invention provides a method of treatment or prophylaxis of AIDS comprising the administration of a vector comprising a sequence of nucleotides encoding a retroviral antigen and a sequence of nucleotides encoding a cytokine or a functional homolog, part, derivative or analogue thereof in conjunction with ARDT wherein said method is effective in maintaining a low retroviral load in a subject or reducing or delaying viral rebound in the absence of ARDT.

To be “effective” an “effective amount” of the instant vector is administered. As used herein an effective amount mean a sufficient amount of the vector to provide the desired therapeutic or physiological outcome. Undesirable effects, e.g. side effects, are sometimes manifested along with the desired therapeutic effect; hence, a practitioner balances the potential benefits against the potential risks in determining what is an appropriate “effective amount”. The exact amount and frequency of administration required will vary from subject to subject, depending on the species, age and general clinical condition of the subject, mode of administration and the like. Thus, it may not be possible to specify an exact “effective amount”. However, an appropriate “effective amount” in any individual case may be determined by one of ordinary skill in the art using only routine experimentation.

The molecules of the present invention can be formulated in pharmaceutic compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. intravenous, oral, intrathecal, epineural or parenteral. Intramuscular administration is preferred.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic or if it would otherwise require too high a dosage or if it would not otherwise be able to enter the desired cells.

Cell based delivery system may be employed such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to cells harbouring latent infection or expression of expression products could be limited to specific cells, stages of development or cell cycle stages. The cell based delivery system is designed to be implanted in a patient's body at the desired site. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

In another aspect, the present invention provides a method of reducing or alleviating one or more of the side effects of ARDT comprising the administration to a subject of a vector comprising a sequence of nucleotides encoding a retroviral antigen and/or a sequence of nucleotides encoding a cytokine, or a functional derivative, homolog, part or analog thereof, for a time and under conditions sufficient to co-express said sequences and to reduce or alleviate one or more of the side effects of ARDT.

In a further aspect, the present invention provides a method of reducing or alleviating one or more of the side effects of ARDT comprising the administration to a subject of a vector comprising a sequence of nucleotides encoding a retroviral antigen and a sequence of nucleotides encoding a cytokine, or a functional derivative, homolog, part or analog thereof, for a time and under conditions sufficient to co-express said sequences and to reduce or alleviate one or more of the side effects of ARDT.

In another aspect, the present invention provides a method of reducing or alleviating one or more of the side effects of ARDT comprising the administration to a subject of a vector comprising a sequence of nucleotides encoding a retroviral antigen and/or a sequence of nucleotides encoding a cytokine, for a time and under conditions sufficient to co-express said sequences and to reduce or alleviate one or more of the side effects of ARDT.

Preferably, said vector is a poxvirus vector. More preferably an avipox vector. Still more preferably a fowlpox vector.

In a further preferred embodiment, the cytokine is IFN-γ.

Preferably the retroviral antigen is gag and/or pol. Most preferably HIV gag/pol is employed.

In a further preferred embodiment, the present invention provides a method of reducing or alleviating one or more of the side effects of ARDT comprising the administration to a subject of a fowlpox vector comprising a sequence of nucleotides encoding HIV gag/pol and a sequence of nucleotides encoding IFN-γ or a functional derivative, homolog, part or analog thereof, for a time and under conditions sufficient to co-express said sequences and to reduce or alleviate one or more side effect of ARDT.

The side effects of ARDT are numerous and are well known in the art and include, without limitation, nausea, vomiting, fever fat redistribution, heart disease, liver disease and insulin resistance. Treatment and prophylaxis regimens are tailored to the individual and include priming and/or boosting with the vector before or during ARDT or after withdrawal ARDT and before or after re-initiation of ARDT. ARDT may be withdrawn for a period of time ranging from days to several months depending on the level and extent of side effects experienced by a recipient and the vector may be administered in prime and/or boost format during this period to maintain low level of viral load. By reducing viral load the methods described herein are useful in increasing the time to restarting ARDT, preventing further ARDT or allowing changes in the combination of anti-retroviral drugs which effectively also reduces the side effects of the treatments.

In a related aspect the present invention extends to the use of the subject vectors in the manufacture of a medicament for use in conjunction with ARDT in the treatment or prophylaxis of a retroviral infection and symptoms associated therewith.

In one aspect, the present invention broadly contemplates the use of a vector comprising a sequence of nucleotides encoding a retroviral antigen and/or a sequence of nucleotides encoding a cytokine or a functional derivative, homolog, part or analog thereof in the manufacture of a medicament for use in a method of reducing or alleviating one or more of the side effects of ARDT.

Preferably the subject has previously been treated with an anti-retroviral compound. The instant vectors may be administering before or during ARDT or after withdrawal of ARDT. When administered before or during ARDT, ARDT may subsequently be withdrawn and in accordance with the present invention, viral loads are maintained at a low level in the absence of ARDT.

In accordance with this aspect of the invention, preferably, said vector is a poxvirus vector. More preferably an avipox vector. Still more preferably a fowlpox vector.

In a further preferred embodiment the cytokine is IFNγ. Preferably the retroviral antigen is gag and/or pol. Most preferably HIV gag/pol are employed.

Accordingly, in a preferred embodiment, the present invention provides the use of a fowlpox vector comprising a sequence of nucleotides encoding HIV gag/pol and a sequence of nucleotides encoding IFNγ or a functional derivative, homolog, part or analog thereof in the manufacture of a medicament for use in a method of reducing or alleviating one or more of the side effects of ARDT.

Said medicament is conveniently in a format for administration as a priming dose and/or a boosting dose. A broad range of doses may be applicable. For example, a unit dose may comprise from about 1×10⁶ PFU per ml to about 1×10⁸ PFU per ml. Dosage regimens are adjusted to provide the optimum therapeutic dose and priming administrations may be administered daily, weekly or monthly or at other suitable time intervals or may be proportionately reduced as indicated by the exigencies of the situation. A preferred priming dose is 5×10⁷ PFU per ml in one ml of diluent. Boosting doses may be the same as priming doses or they may be more or less concentrated as indicated by the exigencies of the situation. For other constructs, from about 0.1 μg to 1 mg of vector may be administered per kilogram of body weight per day.

The present invention is further described by the following non-limiting Examples.

### EXAMPLE 1

### Randomised, Placebo-controlled, Phase I/Ia Evaluation of the Safety and Biological Activity of Avipox Virus Expressing HIV gag-pol and Interferon-gamma in HIV-1 Infected Subjects

A clinical trial was conducted to establish the safety and immunogenicity of recombinant fowlpox virus vaccines (rFPV) expressing HIV gag-pol or co-expressing HIV gag/pol and human interferon-gamma (IFNγ) in HIV positive subjects taking combination anti-retroviral drug therapy (ARDT). A total of 34 patients completed the trial in which they received a series of injections and blood tests regularly over six months. Patients continued to take standard anti-retroviral therapies throughout the trial period. As announced on 17 February, 2003 (virax.com.au) the data for this trial indicated that neither construct elicited a specific immune response in trial participants receiving ARDT.

### EXAMPLE 2

### Safety, Biological Activity and Extension Study to Assess The Anti-retrovirological Properties of a Therapeutic HIV Vaccine Candidate Based on Recombinant Fowlpox Virus (rFPV)

A multicentre, randomised, double-blind, placebo-controlled trial recruited HIV-infected individuals treated with anti-retroviral therapy (ART) during primary HIV infection, who maintained control of virus replication (plasma viral load <50 copies/mL) since initiation of ART. Subjects were randomised to one of three study arms: diluent alone (placebo), rFPV expressing HIV gag/pol (partial construct—PC) or rFPV expressing HIV gag/pol and IFNγ (full construct—FC). Vaccines were administered by intramuscular injection on day 0, week 4 and week 12 at a unit dose of 5×10⁷ pfu/mL in 1.0 mL of diluent. Follow-up continued over 52 weeks. Primary endpoints were mean change in CD8+ effector function as determined by CTL response or ELISPOT assay from baseline to week 26 and increase in log viral load from baseline to week 52. Analyses of safety endpoints was according to treatment received. All analyses were performed using “intention to treat” methods.

In this trial, 35 eligible subjects were randomised (12 placebo, 11 PC-rFPV, 12 FC-rFPV). All but one subject (placebo group) received all three immunizations. All 35 subjects completed 52 weeks of follow-up. No significant toxicity or safety concerns were observed during the study. Episodes of detectable HIV viremia (eight episodes in five patients) were infrequent across the 52 weeks of study and there was no difference between vaccine groups. There were no significant differences between the combined PC and FC groups with placebo patients for anti-HIV gag ELISPOT responses (time-weighted mean difference in change from baseline=−56 sfu/106 PBMC; p=0.062), anti-HIV p55 lymphoproliferative responses (time-weighted mean difference in change from baseline=4.4 SI; p=0.337), anti-HV gag lymphoproliferative responses (time-weighted mean difference in change from baseline=2.1 SI; p=0.778). No additional anti-HIV antibody responses were observed during follow-up. Western Blot reactive anti-FPV antibodies were detected in all PC and FC recipients at week 6 and persisted for the duration of the study. Vaccine recipients generated long-lasting reactive anti-FPV antibodies soon after administration of candidate vaccines.

A pilot multicentre, double-blind, placebo-controlled 20-week extension of the study was conducted to examine the effect of immunisation with recombinant fowlpox virus vaccines (rFPV) on measures of HIV replication following cessation of combination antiretroviral therapy (ART). Previously enrolled individuals protocol were re-consented on day 0, prior to receiving a boosting vaccination by intramuscular injection in accordance with their original randomised assignment: diluent alone (placebo), rFPV expressing HIV gag/pol partial construct—PC) or rFPV expressing HIV gag/pol and interferon-gamma (fill construct—FC). All ART was ceased one week following immunisation. Virological and immunological monitoring was monitored frequently for 20 weeks after immunisation. The primary endpoint was time-weighted area under the curve change from plasma HIV-RNA VL (pVL) at baseline until reintroduction of ART. Secondary endpoints included log pVL after cessation of ART (post-vaccination pVL set-point), kinetics and rate of pVL recrudescence, median time to reinitiation of ART, CD8+ T-cell responses to HIV antigens and CD4+/CD8+ T-cell count changes.

Twenty-five (71%) of the original study cohort consented to participate (placebo=7; PC=8; FC=10). Antiretroviral therapy was re-introduced in 7 patients (placebo=3; PC=3; FC=1). Immunizations were well-tolerated. One patient (PC group) experienced a transient grade 3 thrombocytopenia that resolved without treatment. The time weighted mean change from baseline pVL over 20 weeks was 1.80 (0.72), 1.78 (0.91) and 0.96 (0.91) for placebo, PC and FC respectively (p=0.253, when comparing FC and PC recipients to placebo). The time-weighted mean change from baseline CD4+ cell count was −90.7 (210.1), 2.05 (166.3) and 3.45 (160.9) for placebo, PC and FC respectively (p=0.238, when comparing FC and PC recipients to placebo). All patients had at least one detectable pVL (>50 copies/mL) during follow-up. FC and PC recipients compared to placebo had similar times to detectable pVL (hazard ratio 1.21, 95% CI 0.40-2.97, p=0.682). Time to reinitiation of ART was not statistically significantly different in FC and PC recipients compared to placebo (hazard ratio=2.08, 95% CI 0.49-9.31, p=0.338).

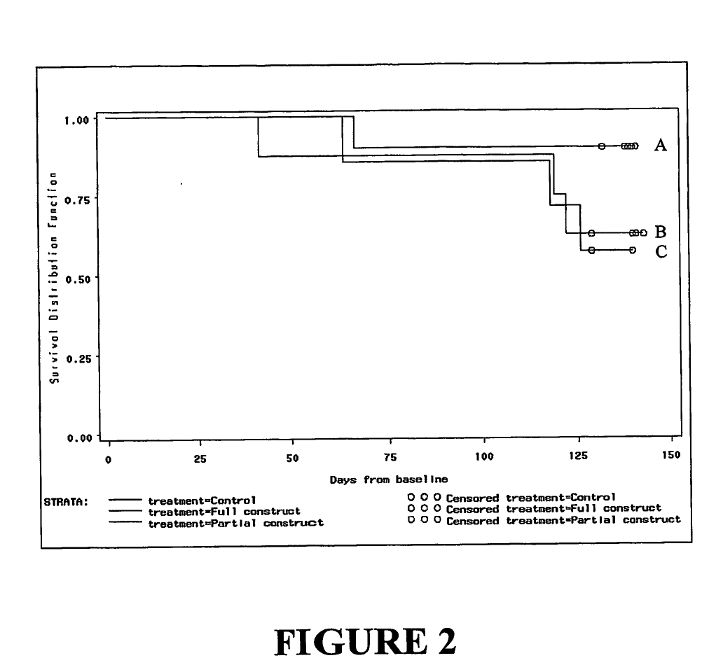

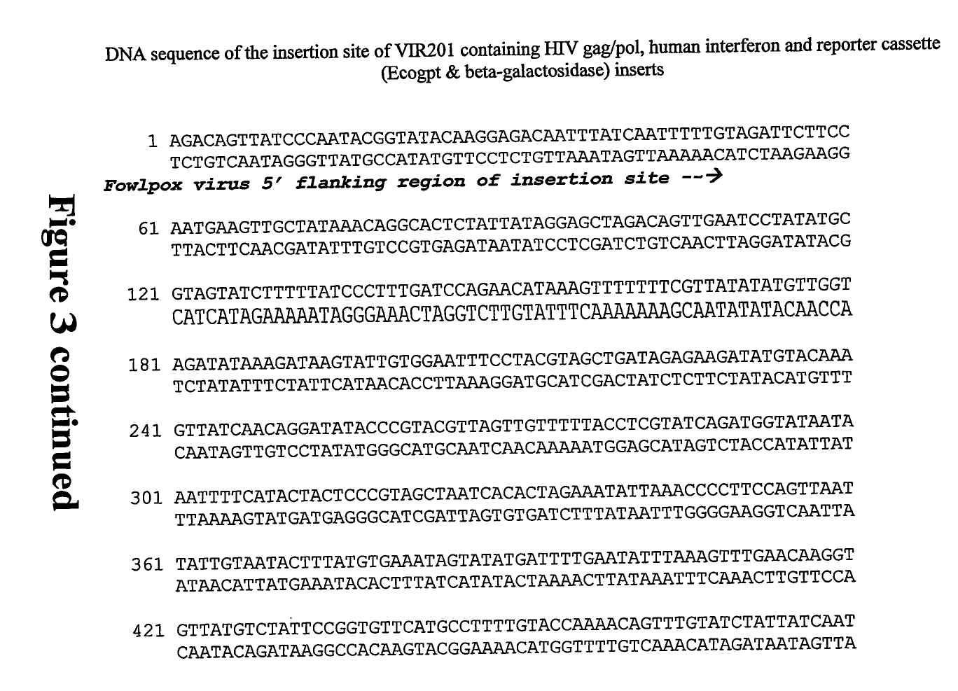

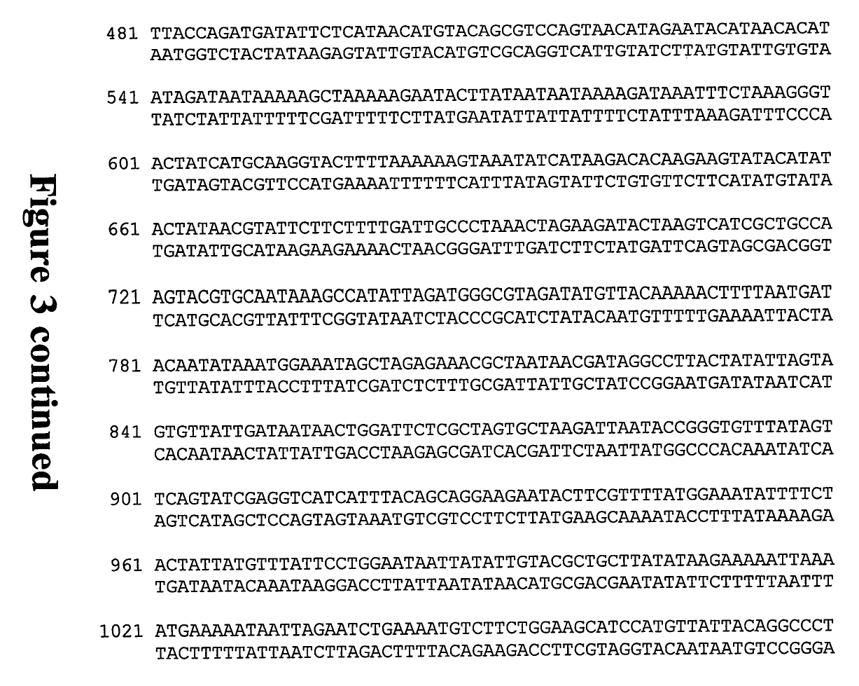

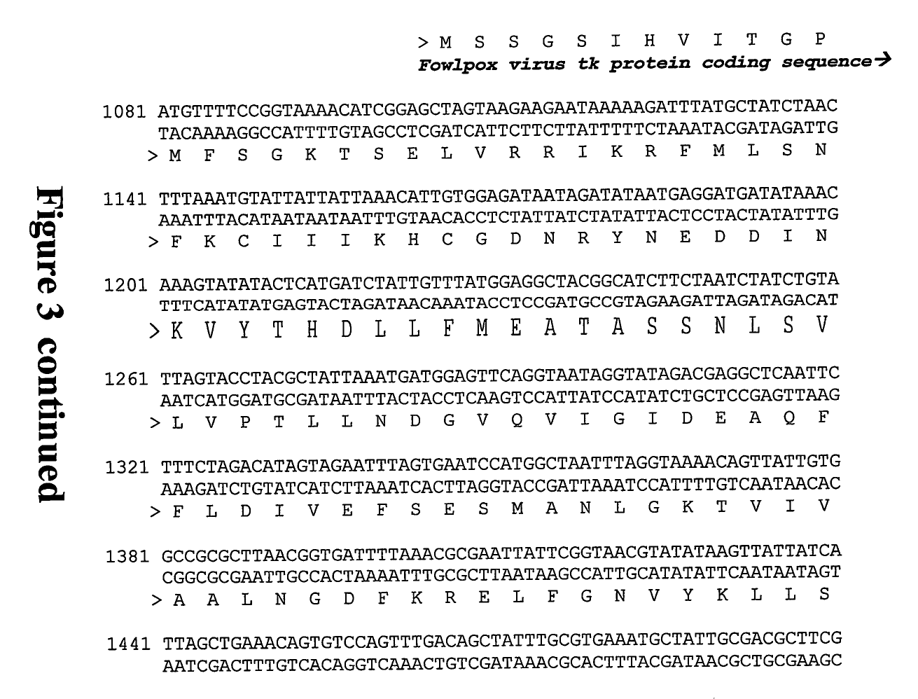

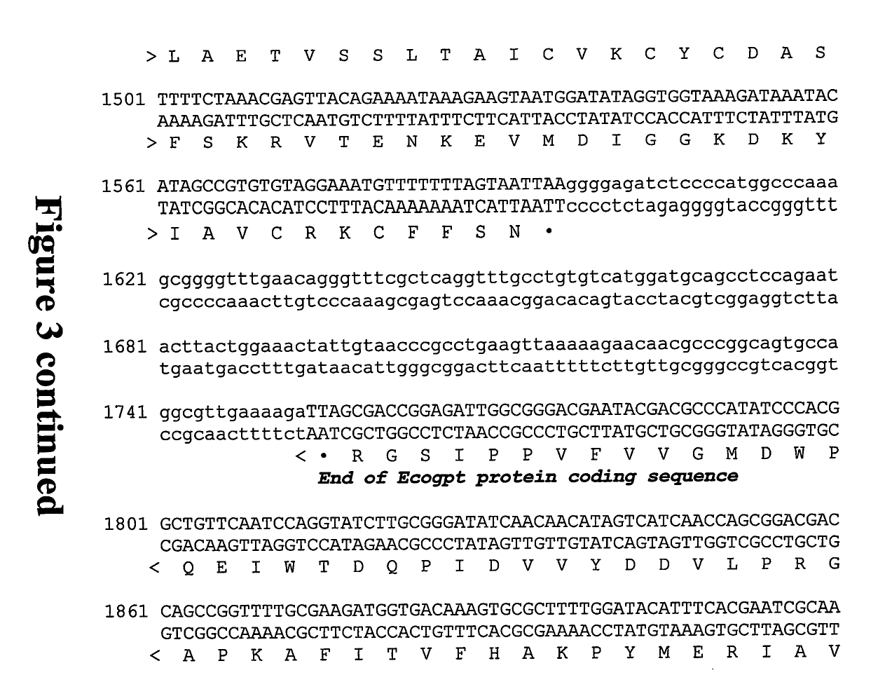

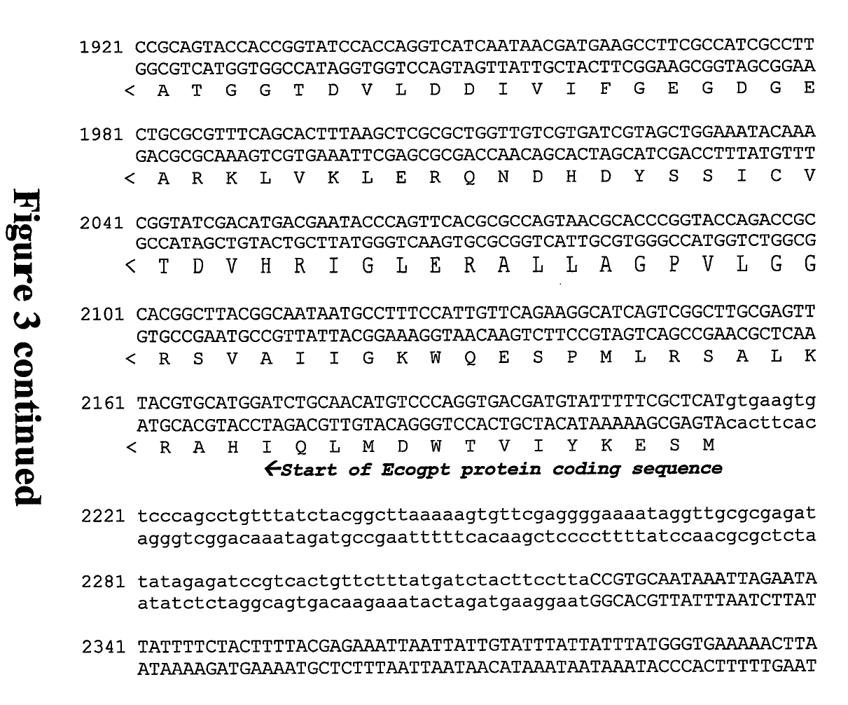

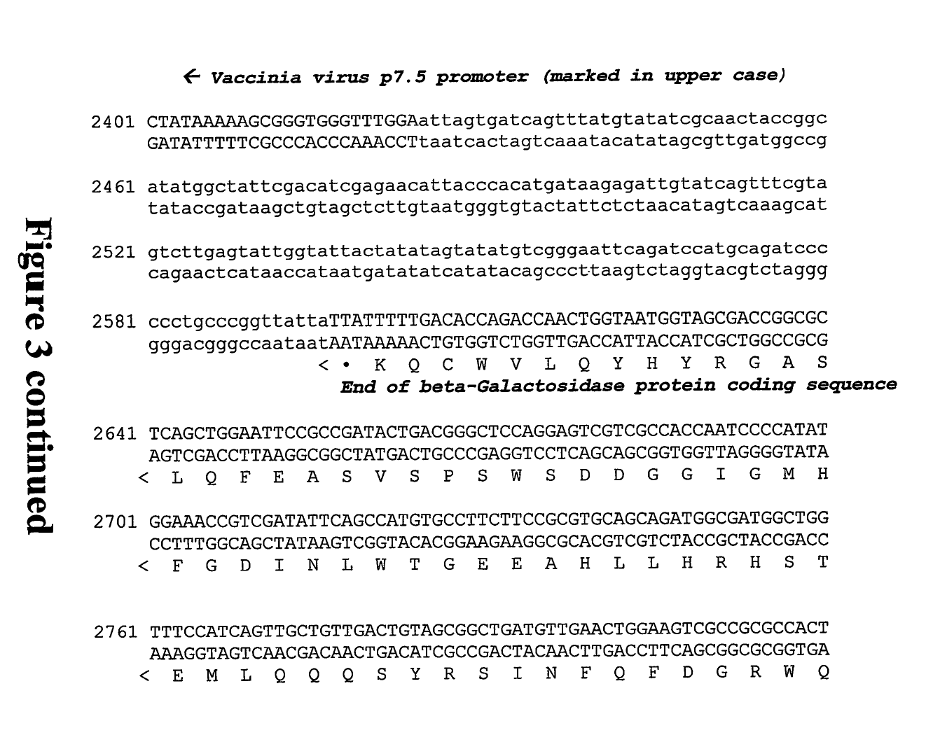

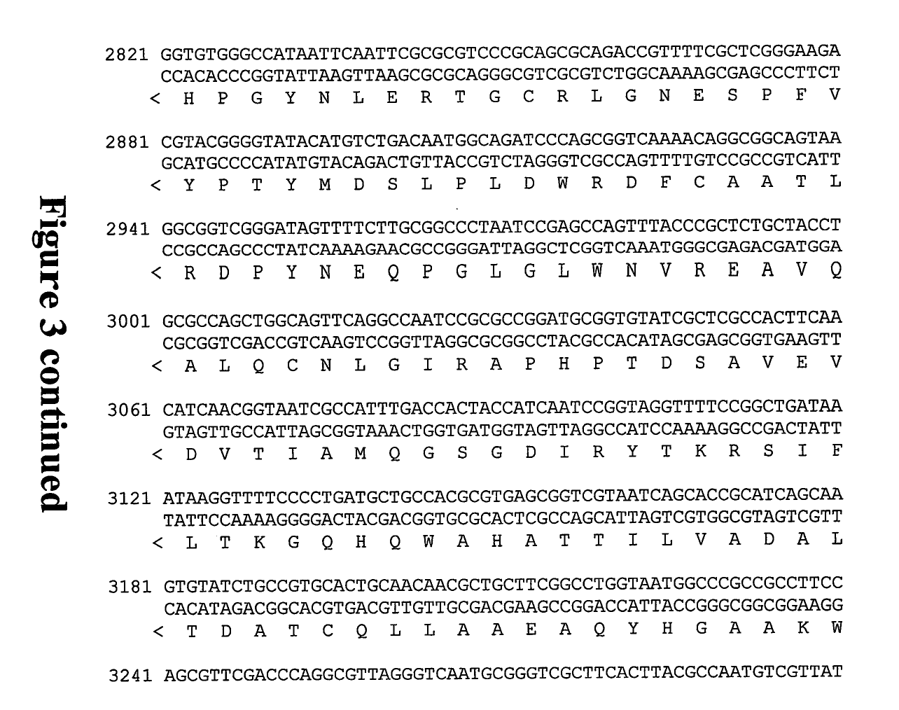

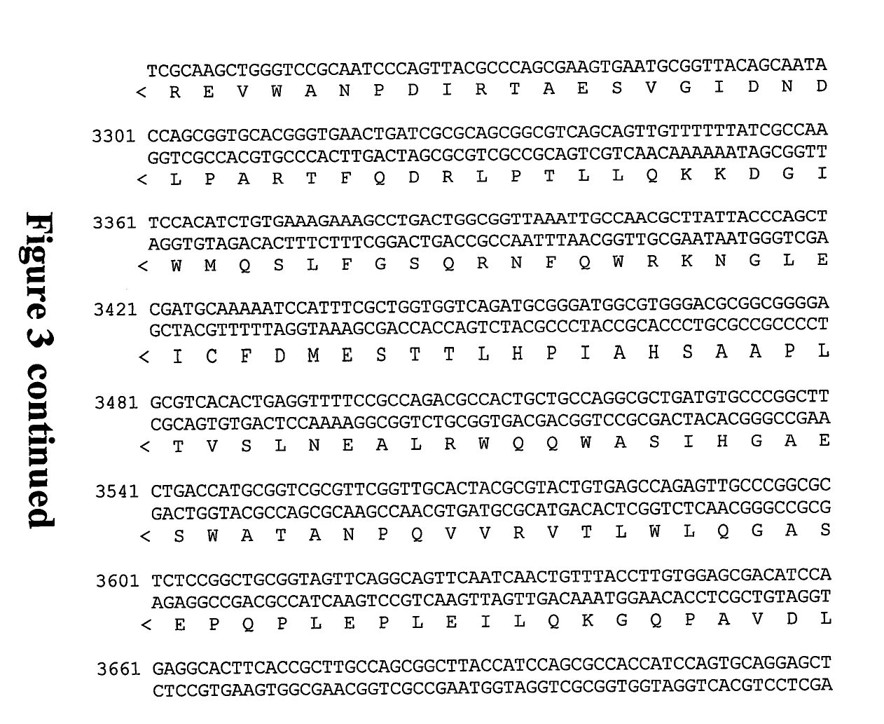

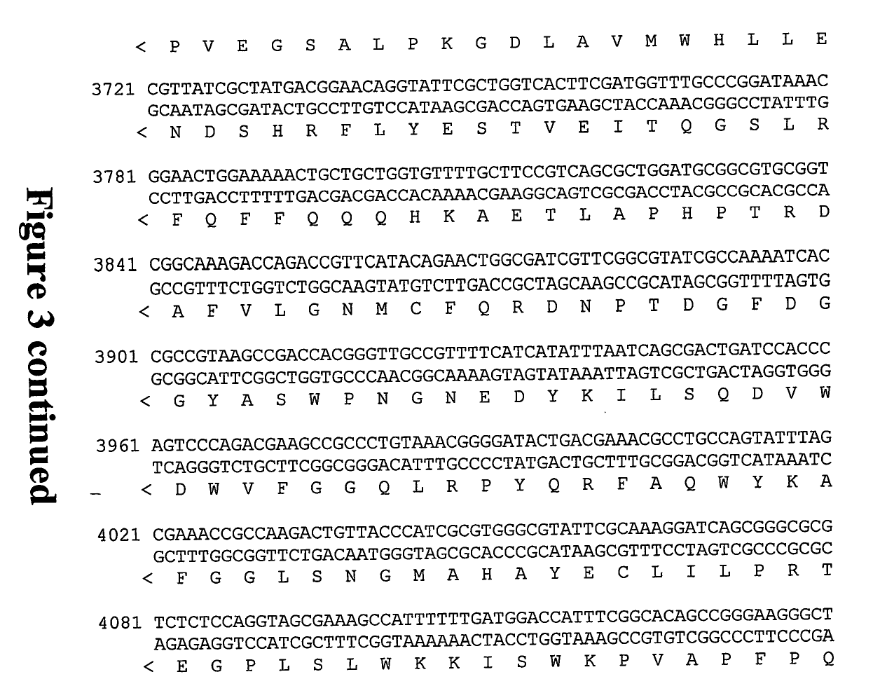

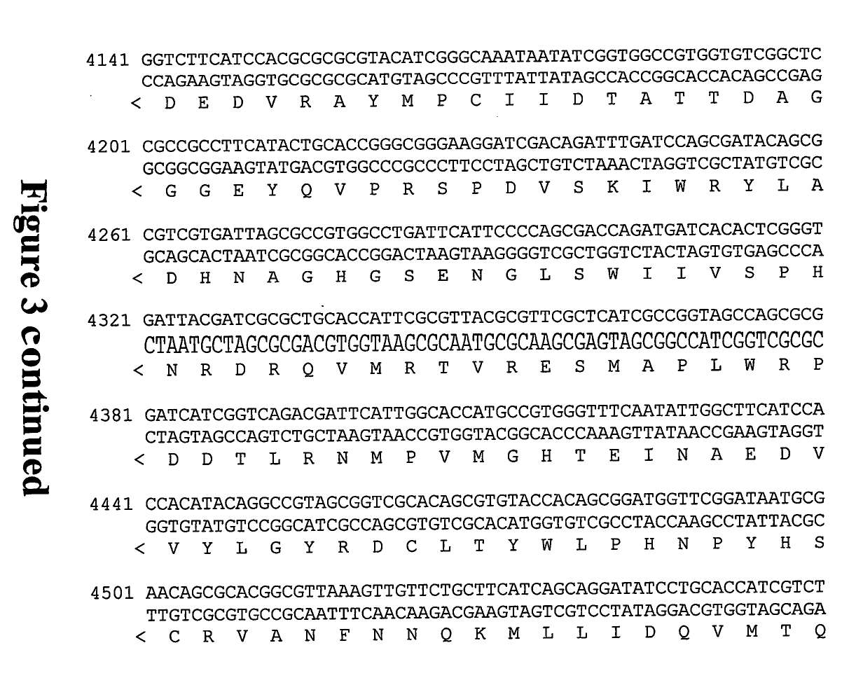

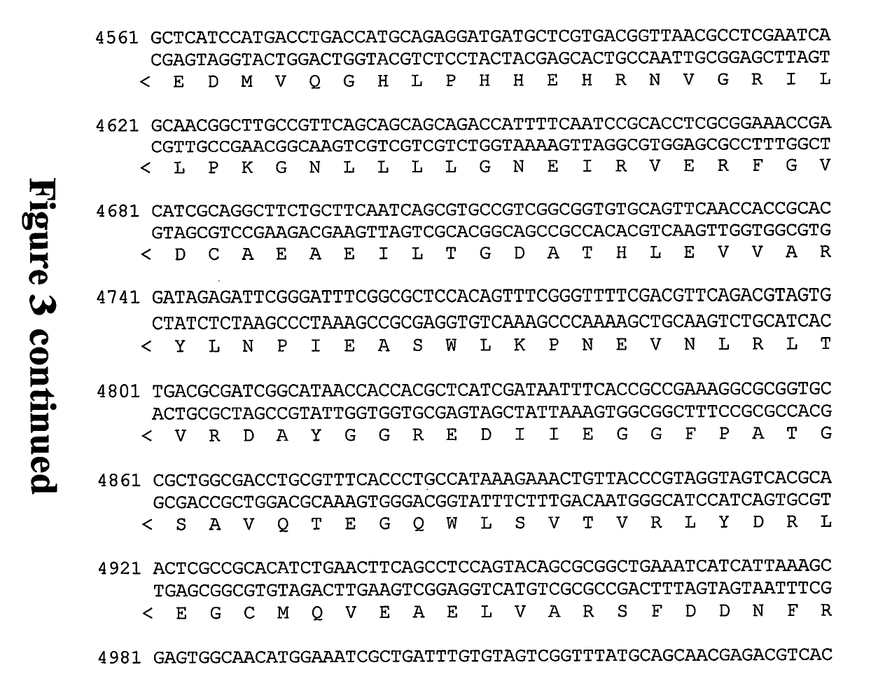

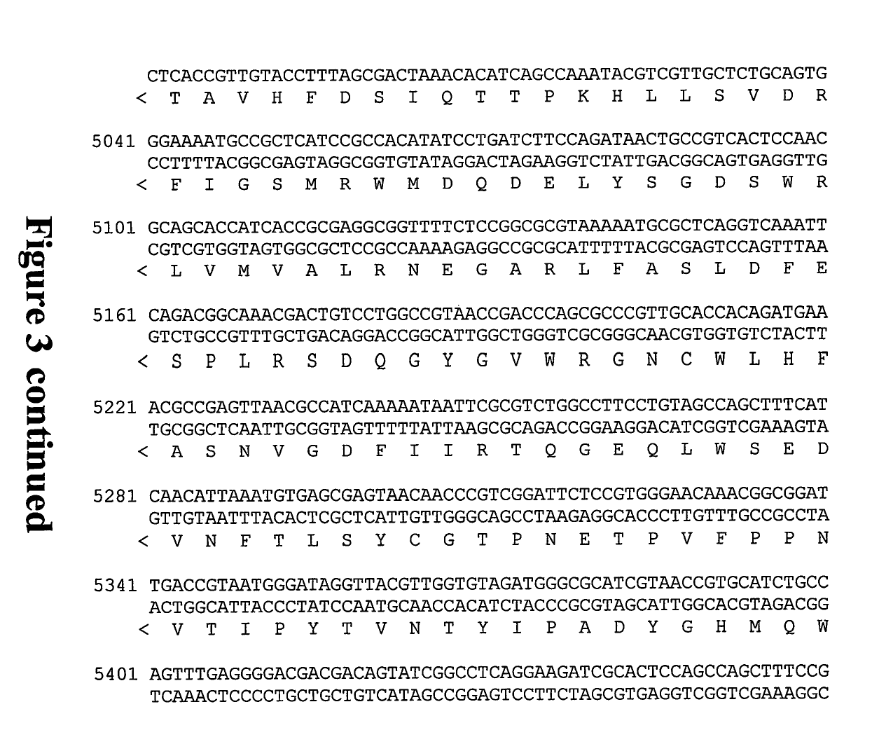

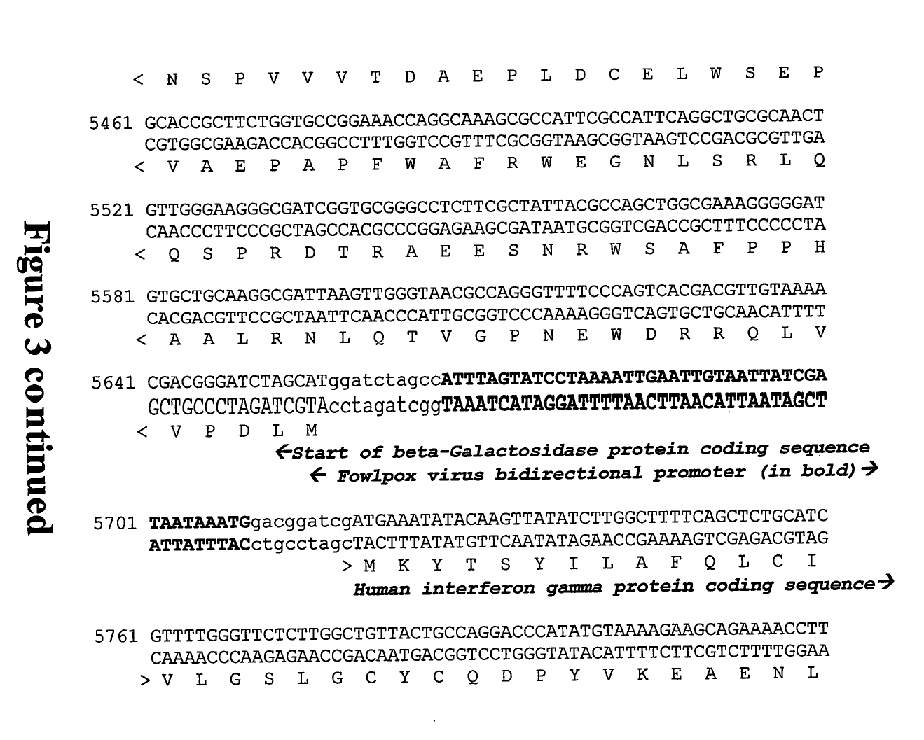

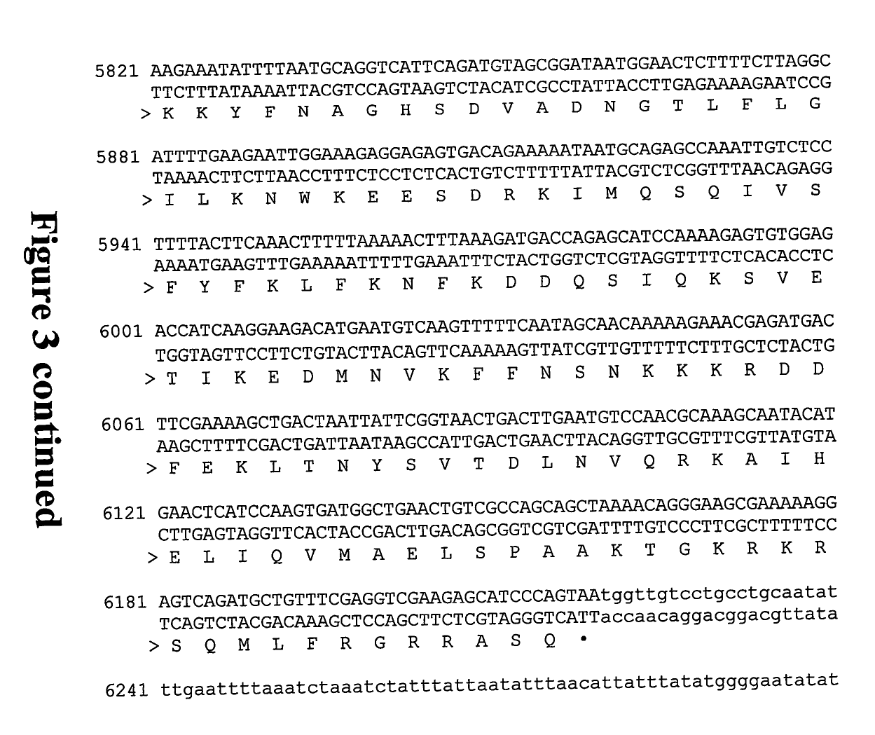

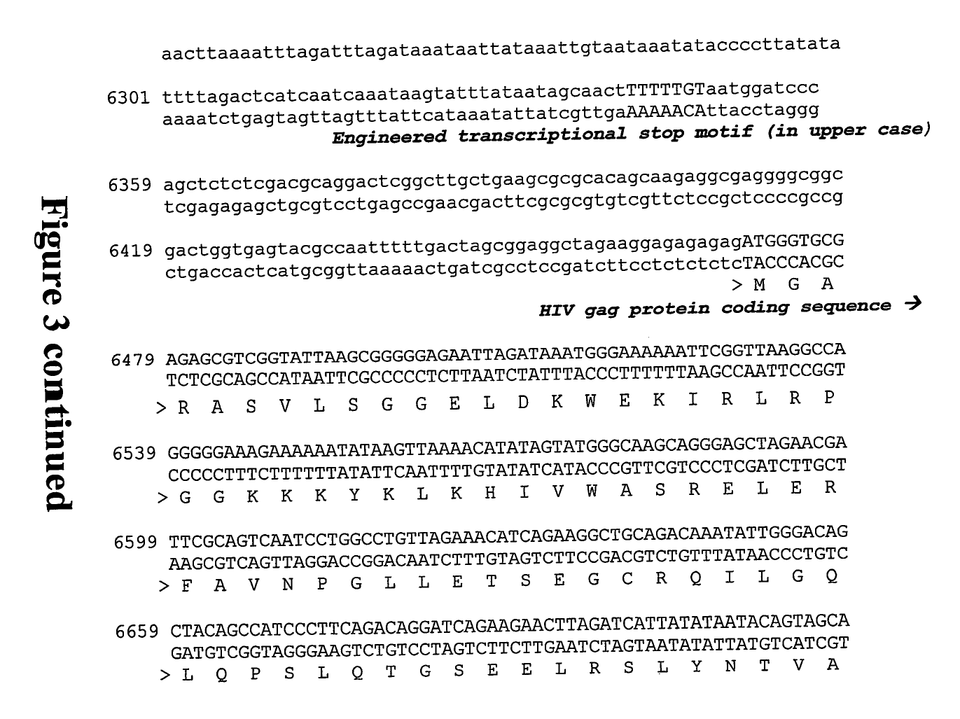

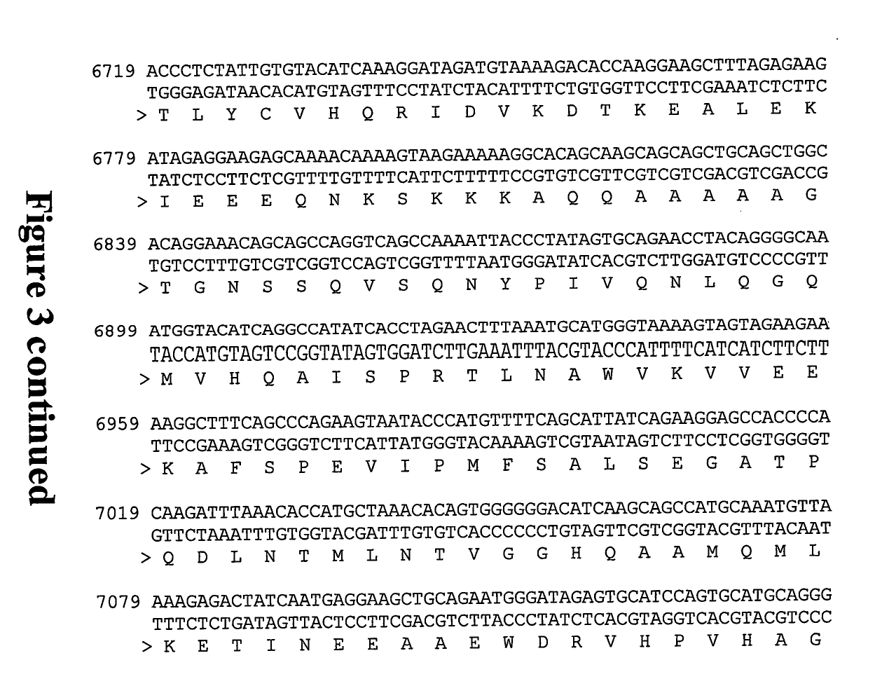

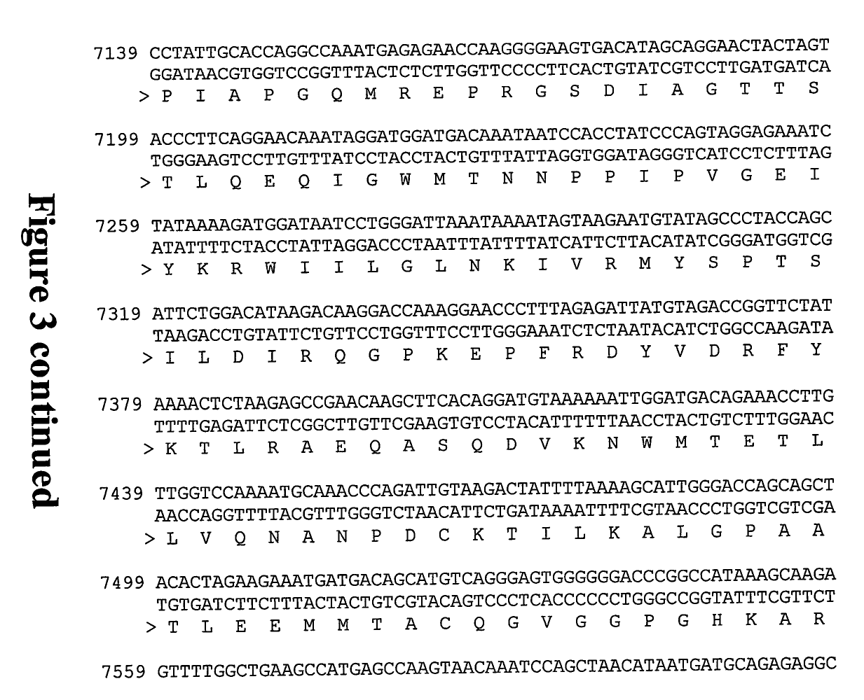

Recipients of the Full construct (FC) rFPV immunization experienced a log reduction in pVL compared to recipients of the PC rFPV or placebo. Specifically, the average maximum viral loads for each of the groups was as follows: placebo group-67173 copies/ml; partial contruct group-68841; and full construct group-18897 (see FIG. 1). Unexpectedly therefore, notwithstanding the lack of any demonstrable immune response in the early part of the trial, in the absence of ARDT, administration of the vector resulted in an approximately 10 fold reduction in average viral load and therapeutic effect over the 20 week period of the study. As specified above, retroviral therapy was re-introduced in a total of seven patients, the seven comprising three from the placebo group, three from the group receiving the partial construct and only one from the largest group receiving the full construct as shown in FIG. 2.

The nucleotide sequence of the insertion site of the vector of rFPV gag/pol IFNγ trialed in this study is set forth in FIG. 3 and is represented in SEQ ID NO: 7.

Those skilled in the art will appreciate that the invention disclosed herein is suceptible to variations and modifications other than those specifically described. It is to be understood that the invention includes all such variations and modifications. The invention also includes all steps, features, compositions referred to or indicated in this specification, individually or collectively, and any and all combinations of any two or more steps or features.

### BIBLIOGRAPHY

Altschul et al., Nucl. Acids Res., 25:3389, 1997.
Australian Society for HIV Medicine (ASHM)-2001, Australian Antiretroviral Guidelines, http://www.ashm.org.au.

### Ausubel et al., “Current Protocols in Molecular Biology” John Wiley & Sons Inc, 1994-1998, Chapter 15.

Bonner, et al., Eur. J. Biochem., 46:83, 1974.

### Harlow and Lare, Antibodies: A Laboratory Manual, Cold Spring Harbor Laboratory, 1998.

Marmur, et al., J. Mol. Biol., 5:109, 1962.

### Melors J. W. et al, Science 272:1167-1170, 1996.

### Paul, Fundamental Immunology, 3^{rd} Ed., 243-247 Raven Press, 1993).

Vogt P. K., Chapter 1 In: Retroviruses, Coffin, J. M.; Hughes, S. H. and Varmus, H. E. (eds.), Cold Spring Harbor Laboratory Press, USA, 1997.

1. A method for the treatment or prophylaxis of a retroviral infection comprising administering to a subject a poxvirus vector encoding an antigen of the retrovirus or the retrovirus antigen and a cytokine, or a functional homolog, derivative, part or analog of the retrovirus antigen and/or the cytokine, in conjunction with anti-retroviral drug therapy wherein the antigen or the antigen and the cytokine are expressed in the subject and are effective in maintaining or prolonging a low retroviral load in the subject for a period of time and are effective in preventing, reducing or delaying viral rebound during interruption of anti-retroviral drug treatment.

2. The method of claim 1, wherein the retroviral infection is HIV infection.

3. The method of claim 1 or 2, wherein the vector is administered to a subject exhibiting a low retroviral viral load as a result of anti-retroviral drug therapy.

4. The method of claim 1 or 2, wherein the vector is administered to a subject exhibiting a low retroviral load prior to commencement of anti-retroviral drug therapy.

5. The method of claim 1, 2, 3 or 4, wherein the cytokine is selected from IFNγ, IL-12, IL-2, TNF and IL-6.

6. The method of claim 5, wherein the cytokine is IFNγ.

7. The method of any one of claims 1 to 6, wherein the retrovirus antigen is encoded by a coding region selected from gag, env, pol and pro coding regions.

8. The method of claim 7, wherein the retrovirus antigen is encoded by gag and/or pol coding regions.

9. The method of claim 8, wherein the retrovirus antigen is encoded by gag and pol coding regions of HIV.

10. The method of any one of claims 1 to 9, wherein the poxvirus vector is an avipox virus vector.

11. The method of claim 10, wherein the avipox virus vector is a fowlpox virus vector.

12. A method for the treatment or prophylaxis of HIV/AIDS comprising administering to a subject a poxvirus vector comprising a sequence of nucleotides encoding a retrovirus antigen and a sequence of nucleotides encoding a cytokine, or a functional homolog, part, derivative or analog of the antigen and/or the cytokine, in conjunction with anti-retroviral drug therapy, wherein said method is effective in maintaining a low retroviral load in the subject and preventing, reducing or delaying retroviral rebound in the absence of anti-retroviral drug therapy.

13. The method of claim 12, wherein the retrovirus antigen is an HIV antigen.

14. The method of claim 12 or 13, wherein the vector is administered to a subject exhibiting a low retroviral viral load as a result of anti-retroviral drug therapy.

15. The method of claim 12 or 13, wherein the vector is administered to a subject exhibiting a low retroviral load prior to commencement of anti-retroviral drug therapy.

16. The method of claim 12, 13, 14 or 15, wherein the cytokine is selected from IFNγ, IL-12, IL-2, TNF and IL-6.

17. The method of claim 16, wherein the cytokine is IFNγ.

18. The method of claim 17, wherein IFNγ comprises the amino acid sequence set forth in SEQ ID NO: 6 or an amino acid sequence having at least about 60% similarity thereto.

19. The method of claim 17, wherein IFNγ is encoded by a sequence of nucleotides set forth in SEQ ID NO: 5 or a sequence of nucleotides encoding a functional homolog, part, derivative or analog thereof having at least 60% similarity thereto, or a sequence which hybridises thereto or to a complementary form thereof under conditions of medium stringency.

20. The method of any one of claims 12 to 19, wherein the retrovirus antigen is encoded by a coding region selected from gag, env, pol and pro coding regions.

21. The method of claim 20, wherein the retrovirus antigen is encoded by gag and/or pol coding regions.

22. The method of claim 21, wherein the retrovirus antigen is encoded by gag and pol coding regions of HIV.

23. The method of claim 22, wherein the retrovirus antigens encoded by gag and pol comprise the amino acid sequence set forth in SEQ ID NO: 2 or a functional homolog, part or derivative thereof or a sequence of amino acids having at least 60% similarity thereto, and SEQ ID NO: 4 or a functional homolog, part or derivative thereof, or a sequence of amino acids having at least 60% similarity thereto, respectively.

24. The method of claim 22, wherein the retrovirus antigen encoded by gag is encoded by a sequence of nucleotides set forth in SEQ ID NO: 1 or a sequence of nucleotides encoding a functional homolog, part or derivative thereof having at least 60% similarity thereto after optimal alignment or a sequence which hybridises thereto or to a complementary form thereof under conditions of medium stringency, and wherein the retrovirus antigen encoded by pol is encoded by a sequence of nucleotides set forth in SEQ ID NO: 3 or a sequence of nucleotides encoding a functional homolog, part or derivative thereof having at least 60% similarity thereto after optimal alignment or a sequence which hybridises thereto or to a complementary form thereof under conditions of medium stringency.

25. The method of any one of claims 12 to 24, wherein the poxvirus vector is an avipox virus vector.

26. The method of claim 25, wherein the avipox virus vector is a fowlpox virus vector.

27. The method of claim 26, wherein the insertion site in the fowlpox vector comprises the sequence of nucleotides set forth in SEQ ID NO: 7.

28. A method of reducing or alleviating one or more side effects of anti-retroviral drug therapy comprising administering to a subject exhibiting a retroviral infection a poxvirus vector comprising a sequence of nucleotides encoding an antigen of the retrovirus or a functional derivative, homolog, part or analog thereof, and a sequence of nucleotides encoding a cytokine or a functional derivative, homolog, part or analog thereof, for a time and under conditions sufficient to co-express the antigen and the cytokine and to reduce or alleviate one or more side effects of anti-retroviral drug therapy in the subject.

29. The method of claim 28, wherein the retroviral infection is HIV infection.

30. The method of claim 28 or 29, wherein the vector is administered to a subject exhibiting a low retroviral viral load as a result of anti-retroviral drug therapy.

31. The method of claim 28 or 29, wherein the vector is administered to a subject exhibiting a low retroviral load prior to commencement of anti-retroviral drug therapy.

32. The method of claim 28, 29, 30 or 31, wherein the cytokine is selected from IFNγ, IL-12, IL-2, TNF and IL-6.

33. The method of claim 32, wherein the cytokine is IFNγ.

34. The method of claim 33, wherein the IFNγ comprises the amino acid sequence set forth in SEQ ID NO: 6 or an amino acid sequence having at least about 60% similarity thereto.

35. The method of claim 33, wherein IFNγ is encoded by a sequence of nucleotides set forth in SEQ ID NO: 5 or a sequence of nucleotides encoding a functional homolog or derivative thereof having at least 60% similarity thereto, or a sequence which hybridises thereto or to a complementary form thereof under conditions of medium stringency.

36. The method of any one of claims 28 to 35, wherein the retrovirus antigen is encoded by a coding region selected from gag, env, pot and pro coding regions.

37. The method of claim 36, wherein the retrovirus antigen is encoded by gag and/or pol coding regions.

38. The method of claim 37, wherein the retrovirus antigen is encoded by gag and pot coding regions of HIV.

39. The method of claim 38, wherein the retrovirus antigens encoded by gag and pol comprise the amino acid sequence set forth in SEQ ID NO: 2 or a functional homolog, part or derivative thereof, or a sequence of amino acids having at least 60% similarity thereto, and SEQ ID NO: 4 or a functional homolog, part or derivative thereof, or a sequence of amino acids having at least 60% similarity thereto, respectively.

40. The method of claim 38, wherein the retrovirus antigen encoded by gag is encoded by a sequence of nucleotides set forth in SEQ ID NO: 1 or a sequence of nucleotides encoding a functional homolog, part or derivative thereof, having at least 60% similarity thereto after optimal alignment, or a sequence which hybridises thereto or to a complementary form thereof under conditions of medium stringency, and wherein the retrovirus antigen encoded by pol is encoded by a sequence of nucleotides set forth in SEQ ID NO: 3 or a sequence of nucleotides encoding a functional homolog, part or derivative thereof having at least 60% similarity thereto after optimal alignment, or a sequence which hybridises thereto or to a complementary form thereof under conditions of medium stringency.

41. The method of any one of claims 28 to 40, wherein the poxvirus vector is an avipox virus vector.

42. The method of claim 41, wherein the avipox virus vector is a fowlpox virus vector.

43. The method of claim 42, wherein the insertion site in the fowlpox vector comprises the sequence of nucleotides set forth in SEQ ID NO: 7.

44. A use of a recombinant vector comprising a sequence of nucleotides encoding a retrovirus antigen or a functional derivative, homolog, part or analog thereof, and a sequence of nucleotides encoding a cytokine or a functional derivative, homolog, part or analog thereof in the manufacture of a medicament for use in maintaining or prolonging a low retroviral load in a subject for a period of time, and in preventing, reducing or delaying viral rebound during interruption of anti-retroviral drug treatment.

45. A use of a recombinant vector comprising a sequence of nucleotides encoding a retrovirus antigen or a functional derivative, homolog, part or analog thereof, and a sequence of nucleotides encoding a cytokine or a functional derivative, homolog, part or analog thereof, in the manufacture of a medicament for use in reducing or alleviating one or more side effects of anti-retroviral drug therapy.

46. A use according to claim 44 or 45, wherein the retrovirus is HIV.

47. A recombinant poxvirus vector comprising a sequence of nucleotides encoding a retrovirus antigen or a functional homolog, derivative, part or analog thereof, and a sequence of nucleotides encoding a cytokine or a functional homolog, derivative, part or analog thereof, for use in conjunction with anti-retroviral drug therapy to maintain or prolong a low retroviral load in a subject and to prevent, reduce or delay viral rebound during interruption of anti-retroviral drug treatment in a subject.

48. A recombinant pol virus vector comprising a sequence of nucleotides encoding a retrovirus antigen or a functional homolog, derivative, part or analog thereof, and a sequence of nucleotides encoding a cytokine or a functional homolog, derivative, part or analog thereof, for use in reducing or alleviating one or more side effects of anti-retroviral drug therapy.

49. The recombinant poxvirus vector of claim 48, wherein the for use in maintaining or prolonging a low retroviral load in the subject and reducing or alleviating one or more side effects of anti-retroviral drug therapy.

50. The recombinant poxvirus vector of claims 47, 48 or 49, wherein the retrovirus is HIV.

51. The recombinant vector of claims 47, 48, 49 or 50, wherein the cytokine is selected from IFNγ, IL-12, IL-2, TNF and IL-6.

52. The recombinant vector of claim 51, wherein the cytokine is IFNγ.

53. The recombinant vector of claim 52, wherein the IFNγ comprises the amino acid sequence set forth in SEQ ID NO: 6 or an amino acid sequence having at least about 60% similarity thereto.

54. The recombinant vector of claim 52, wherein IFNγ is encoded by a sequence of nucleotides set forth in SEQ ID NO: 5 or a sequence of nucleotides encoding a functional homolog or derivative thereof having at least 60% similarity thereto or a sequence which hybridises thereto or to a complementary form thereof under conditions of medium stringency.

55. The recombinant vector of any one of claims 47 to 54, wherein the retrovirus antigen is encoded by a coding region selected from gag, env, pol and pro coding regions.

56. The recombinant vector of claim 55, wherein the retrovirus antigen is encoded by gag and/or pol coding regions.

57. The recombinant vector of claim 56, wherein the retrovirus antigen is encoded by gag and pol coding regions of HIV.

58. The recombinant vector of claim 57, wherein the retrovirus antigens encoded by gag and pol comprise the amino acid sequence set forth in SEQ ID NO: 2 or a functional homolog, part or derivative thereof or a sequence of amino acids having at least 60% similarity thereto, and SEQ ID NO: 4 or a functional homolog, part or derivative thereof or a sequence of amino acids having at least 60% similarity thereto, respectively.

59. The recombinant vector of claim 57, wherein the retrovirus antigen encoded by gag is encoded by a sequence of nucleotides set forth in SEQ ID NO: 1 or a sequence of nucleotides encoding a functional homolog, part or derivative thereof having at least 60% similarity thereto after optimal alignment or a sequence which hybridises thereto or to a complementary form thereof under conditions of medium stringency, and wherein the retrovirus antigen encoded by pol is encoded by a sequence of nucleotides set forth in SEQ ID NO: 3 or a sequence of nucleotides encoding a functional homolog, part or derivative thereof having at least 60% similarity thereto after optimal alignment or a sequence which hybridises thereto or to a complementary form thereof under conditions of medium stringency.

60. The recombinant vector of any one of claims 47 to 59, wherein the poxvirus vector is an avipox virus vector.

61. The recombinant vector of claim 60, wherein the avipox virus vector is a fowlpox virus vector.

62. The recombinant vector of claim 61, wherein the insertion site in the fowlpox vector comprises the sequence of nucleotides set forth in SEQ ID NO: 7.
